(19) United States
(12) Patent Application Publication
(10) Pub. No.: US 2026/0184740 A1
(43) Pub. Date: Jul. 2, 2026

(54) Semi-Automated High-Throughput Mid-Scale Protein Expression Methods

(71) Applicant: Genentech, Inc., South San Francisco, CA (US)

(72) Inventors: Kanika Bajaj, Cupertino, CA (US); Inna Zilberleyb, Foster City, CA (US); Yvonne Christa Franke, Mountain View, CA (US); Edward Allan Kraft, Holladay, UT (US)

(73) Assignee: Genentech, Inc., South San Francisco, CA (US)

(21) Appl. No.: 19/552,288

(22) Filed: Feb. 27, 2026

Related U.S. Application Data
(63) Continuation of application No. PCT/US2024/044170, filed on Aug. 28, 2024.
(60) Provisional application No. 63/579,668, filed on Aug. 30, 2023.
(60) Provisional application No. 63/645,754, filed on May 10, 2024.

(51) Int. Cl. C07K 1/34; C07K 1/22; C07K 1/36

(52) U.S. Cl. CPC C07K 1/34; C07K 1/22; C07K 1/36

(57) ABSTRACT

The present disclosure relates to a higher-throughput, mid-scale, semi-automated protein expression and screening platform that can be used, for instance, for drug discovery research and otherwise for testing protein expression conditions, among other uses. The workflow described here also in some embodiments enables comprehensive expression and purification screening assessment of challenging or difficult-to-express recombinant proteins in a faster and efficient manner by delivering small but sufficient amounts of high-quality proteins.

### CROSS REFERENCE TO RELATED APPLICATIONS

This application is a continuation of International Application No. PCT/US2024/044170, filed Aug. 28, 2024, which claims priority to U.S. Application No. 63/579,668, filed Aug. 30, 2023, and U.S. Application No. 63/645,754, filed May 10, 2024, the entire contents of which are incorporated by reference herein for all purposes.

### FIELD

The present disclosure relates to a higher-throughput, mid-scale, semi-automated protein expression and screening platform that can be used, for instance, for drug discovery research and otherwise for testing protein expression conditions, among other uses. The workflow described here also in some embodiments enables comprehensive expression and purification screening assessment of challenging or difficult-to-express recombinant proteins in a faster and efficient manner by delivering small but sufficient amounts of high-quality proteins.

### BACKGROUND

Recombinant protein production is a critical component of basic and drug discovery research. As therapeutic targets become more complex, researchers are continuously finding innovative expression and purification technologies to improve protein production of difficult-to-express proteins and challenging multiprotein complexes in a faster and more cost-effective way. High-throughput expression analysis at small-scale, while fast and efficient for triaging most types of protein targets, often provides insufficient protein yield and information for challenging targets, which necessitates costly large-scale production for additional characterization and poses a significant bottleneck for drug discovery research. Researchers often require production of tens to hundreds of protein variants to tackle such complex drug discovery targets which is both resource and time intensive. This necessitates building efficient and effective upstream screening strategies to narrow down optimal construct and expression and purification conditions in order to support such targets.

Moreover, the classical toolbox for drug discovery is also expanding beyond traditional small and large molecules. New emerging therapeutic modalities—such as degraders, macrocycle peptides (MCP), and disulfide constrained peptides (DCP)—may be pursued in parallel at an early stage to address undruggable targets that often require milligram quantities of high-quality proteins. One exemplary expression approach is to generate and screen a series of constructs using multiple expression systems in order to identify the most suitable construct and system for the generation of sufficient quantities of stable and functionally active protein for structural and functional studies. Multiple laboratories have built automation and bioinformatics tools to enable high-throughput small scale protein expression analysis platforms to allow rapid triaging of multiple constructs in parallel in different expression systems (Esposito, Garvey, & Chakiath, 2009; Marsischky & LaBaer, 2004; Chambers, Austen, Fulghum, & Kim, 2004; Festa, Steel, Bian, & Labaer, 2013; Gileadi et al., 2008; Hunt, 2005; Kraft et al., 2019). However, the small-scale approach suffers from certain drawbacks. For example, it offers limited flexibility and screening options to address poorly expressing proteins and multiprotein complexes, particularly those that have unstructured domains, are aggregation prone and need additional co-expression partners to express, fold and function properly.

### SUMMARY

Accordingly, the present application provides new methods for high-throughput expression of proteins on a larger scale, and which may address drawbacks associated with prior, smaller-scale approaches. For example, methods described herein address the limitations of small scale approaches and to provide a faster and effective higher throughput triaging solution for screening optimal expression and purification conditions for challenging target proteins. Methods herein, in some embodiments, allow for parallel expression, purification, and characterization of, for example, from a dozen to about a hundred samples, from cell culture, to identify optimal constructs and suitable conditions for poorly expressing proteins, assess co-expression partners in multiprotein complexes, and enable buffer/additive screening for insoluble and aggregation-prone proteins, among other uses. In addition to protein screening, methods herein also provide small but sufficient amounts of high-quality proteins for desired downstream application screening, e.g., negative stain, biochemical activity assay, DNA binding assay, affinity pull-downs, DNA Encoded Chemical Library (DEL) screens, and Surface Plasmon Resonance (SPR) screens, among many others. The methods herein can also be widely adopted with minimal protocol modifications for all types of targets and expression systems, including intracellular, secreted, and membrane-bound proteins expressed in E. coli, BEVS, and mammalian systems. The methods may also be used for expressing full-length proteins, specific domains, mutant proteins, or chimeric proteins, and can be used with various peptide affinity tags or fusion partners, such as polyhistidine, FLAG, glutathione-S-transferase (GST), and maltose binding protein (MBP), and others (see Kimple, Brill, & Pasker, 2013). Furthermore, methods herein may provide an efficient construct triaging platform in multiple expression systems in parallel for moderate to high expressing properly folded single subunit proteins. For example, gel analysis can be used in some cases to confirm the presence of full-length or truncated proteins, identify the molecular weight, and provide information regarding sample purity. Additionally, methods herein may be used to provide recommendations for the best expression system, cell line, and tagging strategy to express a particular protein or domain successfully at large scale.

Exemplary embodiments herein include the following:

1. A method for purifying one or more polypeptides from a plurality of mammalian cell culture samples, insect cell culture samples, and/or bacterial cell culture samples, comprising:
(a) growing a plurality of mammalian, insect, or bacterial cell culture samples expressing one or more polypeptides for purification at a scale of 20-500 mL;
(b) if necessary to separate the expressed polypeptide(s) from cell debris, such as where the polypeptides are not secreted by the cells, lysing the cells of the cell culture samples, optionally wherein parts (a) and (b) for each of the plurality of cell culture samples are conducted in the same container;
(c) centrifuging the lysed cell culture samples and collecting a plurality of supernatant samples from the cell culture samples;
(d) clarifying the plurality of supernatant samples, optionally by filtration such as depth filtration;
(e) placing the plurality of clarified supernatant samples into wells of a multi-well plate, wherein the supernatant samples have a volume of 2-30 mL per well; and
(f) subjecting the plurality of supernatant samples in the wells of the multi-well plate to affinity chromatography conducted with an affinity matrix in a pipette tip and placing the eluate from the chromatography into wells of a multi-well plate (i.e., the same or a different multi-well plate); wherein part (f) or parts (e) and (f) are conducted on the plurality of samples in parallel.

2. The method of embodiment 1, wherein the following parts the method are automated: part (f), or parts (e) and (f).

3. The method of embodiment 1, wherein parts (d)-(f), parts (e) and (f), or all of parts (a)-(f) are performed in parallel.

4. The method of any one of embodiments 1-3, wherein the plurality of clarified supernatant samples are placed into wells of the multi-well plate at a volume of 2-30 mL per well.

5. A method for purifying one or more polypeptides starting from a plurality of clarified cell culture supernatant samples in a multi-well plate, which were obtained from a plurality of cell culture samples by a process comprising:
(a) growing a plurality of mammalian, insect, and/or bacterial cell culture samples expressing one or more polypeptides for purification at a scale of 20-500 mL,
(b) if necessary to separate the expressed polypeptide(s) from cell debris, such as where the polypeptides are not secreted by the cells, lysing the cells of the cell culture samples, optionally wherein parts (a) and (b) for each of the plurality of cell culture samples are conducted in the same container;
(c) centrifuging the lysed cell culture samples and collecting a plurality of supernatant samples from the cell culture samples;
(d) clarifying the plurality of supernatant samples, optionally by filtration such as by depth filtration;
(e) placing the plurality of clarified supernatant samples into wells of the multi-well plate, wherein the supernatant samples have a volume of 2-30 mL per well;
the method comprising: (f) subjecting the supernatant samples obtained from the process of (a)-(e) to affinity chromatography conducted with an affinity matrix in a pipette tip and placing the eluate from the chromatography into wells of a multi-well plate; wherein, the method is conducted on the plurality of clarified supernatant samples in parallel, and optionally wherein the method is automated.

6. A method for affinity purification of polypeptides from a plurality of cell culture supernatant samples expressing one or more polypeptides for purification, comprising: obtaining a multi-well plate comprising a plurality of clarified cell culture supernatant samples in wells of the multi-well plate at a volume of 2-30 mL per well, and subjecting the plurality of clarified cell culture supernatant samples to affinity chromatography conducted with an affinity matrix in a pipette tip and placing the eluate from the chromatography into wells of a second multi-well plate; wherein, the method is conducted on the plurality of supernatant samples in parallel, optionally wherein the method is automated, wherein the plurality of cell culture supernatant samples were obtained from mammalian, insect, and/or bacterial cell culture samples expressing one or more polypeptides, which were grown at a scale of 20-500 mL and subjected to one or more of lysis, centrifugation, and clarification, optionally wherein growth and lysis of the cells were conducted in the same container.

7. The method of any one of embodiments 1-6, wherein the method further comprises:
(g) conducting size exclusion chromatography (SEC) on the eluate of the affinity chromatography; and
(h) fractionating polypeptides from the SEC into wells of a multi-well plate, optionally wherein one or both of parts (g) and (h) is automated.

8. The method of embodiment 7, wherein parts (g) and (h) are automated.

9. The method of any one of embodiments 1-8, wherein clarified supernatant sample corresponding to a single cell culture sample is placed into more than one well of the multi-well plate.

10. The method of any one of embodiments 1-9, wherein clarified supernatant samples from different cell culture samples are placed into different wells of the multi-well plate.

11. The method of any one of embodiments 1-10, wherein from 8 to 96 clarified supernatant samples are processed in parallel, such as from 8 to 48, from 8 to 24, from 12 to 48, or from 12 to 24.

12. The method of any one of embodiments 1-11, wherein cells are lysed by addition of glass beads with shaking and/or wherein the cells are not lysed by sonication.

13. The method of any one of embodiments 1-12, wherein the plurality of cell culture samples are grown at a scale of 30-250 mL, 50-250 mL, 30-200 mL, 50-200 mL, or 100-200 mL.

14. The method of any one of embodiments 1-13, wherein the pipette tip comprising the affinity matrix has a volume of 0.5 to 2 mL, such as from 1 to 2 mL, or 0.5 to 1.5 mL, or 0.5 mL, or 1 mL, or 1.5 mL, or 2 mL.

15. The method of any one of embodiments 1-14, wherein the affinity matrix in the pipette tip has a bed volume of from 30 to 100 μL, such as from 30 to 70 μL, or from 40 to 50 μL, or 30 μL, 40 μL, 50 μL, 60 μL, 70 μL, or 100 μL.

16. The method of any one of embodiments 1-15, wherein the polypeptides are tagged with a polyhistidine, FLAG, streptavidin, glutathione-S-transferase (GST), or maltose binding protein (MBP) tag, and wherein the affinity matrix recognizes the tag.

17. The method of any one of embodiments 7-16, wherein the method comprises parts (g) and (h), and wherein the SEC chromatography is performed on an SEC matrix comprising particles with pore size between 140 and 500 Angstroms, and/or with a particle size of 3 to 5 microns, and/or with a molecular weight range of 5 to 700 kDa.

18. The method of any one of embodiments 1-17, wherein the method further comprises performing structural or functional analysis on the purified polypeptides, such as cryoelectron microscopy, mass spectrometry, protein-protein interaction assays such as surface plasmon resonance, or homogeneous time resolved fluorescence assays.

19. The method of any one of embodiments 1-18, wherein the one or more polypeptides comprise recombinant protein complexes.

20. The method of any one of embodiments 1-19, wherein the one or more polypeptides do not comprise antibodies or antibody subunits.

21. The method of any one of embodiments 1-20, wherein the cell culture samples are insect cell culture samples, such as Sf9 or T.ni cell culture samples.

22. The method of any one of embodiments 1-21, wherein the cell culture samples are mammalian cell culture samples, such as HEK293 or CHO cells.

23. A system for performing the method of any one of embodiments 1-22, wherein the system comprises a means of performing automated affinity chromatography on a plurality of cell culture samples of 2-30 mL volume in parallel, wherein the samples are located in wells of a multi-well plate (e.g., of 8-96 wells), wherein the chromatography is performed using an affinity matrix in a pipette tip, and the system also comprises a means of placing the eluate from the chromatography into wells of a multi-well plate (such as a second multi-well plate).

24. A kit comprising reagents for performing a method of any one of claims 1-22, wherein the kit optionally comprises one or more of: reagents for performing affinity chromatography on a plurality of 2-30 mL samples in parallel using an affinity matrix in a pipette tip (e.g., tips comprising the affinity matrix, and/or one or more buffers such as equilibration, wash, and elution buffers), reagents for performing SEC chromatography on the samples (e.g., SEC columns, and/or one or more buffers such as equilibration, wash, and elution buffers), a positive control sample, a negative control sample, one or more multi-well plates for holding samples, and instructions for use.

Additional objects and advantages will be set forth in part in the description which follows, and in part will be understood from the description, or may be learned by practice. The objects and advantages will be realized and attained by means of the elements and combinations particularly pointed out in the appended claims. It is to be understood that both the foregoing general description and the following detailed description are exemplary and explanatory only and are not restrictive of the claims.

The accompanying drawings, which are incorporated in and constitute a part of this specification, illustrate certain embodiments and together with the description, serve to further explain certain principles described herein.

### BRIEF DESCRIPTION OF THE DRAWINGS

The present US provisional application includes at least one drawing executed in color. In the event that a nonprovisional or PCT application claiming priority to this US provisional application and incorporating the contents of this provisional application publishes in the future, copies of this provisional patent application including the color drawings will be provided by the Office upon request and payment of the necessary fee.

FIG. 1. Mid-scale expression and purification steps. Schematic workflow describing the steps involved in mid-scale expression and purification of recombinant proteins: Insect cell cultures are grown in 500 mL conical tubes; cells are lysed using glass beads and clarified by depth filtration; affinity purification is performed in 24-well plates using IMCS tips; proteins are further purified and characterized through size exclusion chromatography and analysis by SDS-PAGE. Proteins purified from this workflow are used for several downstream applications, including negative stain and biochemical assays.

FIG. 2. Guide on visual interpretation of flow cytometry results. Examples of successful and unsuccessful infections based on GP64 positive and viable cell populations.

FIG. 3. Lysate layout for depth filtration. Cell lysates are clarified using centrifugation and depth filtration process. Orochem 24-well depth filters are placed on top of 24-well deep well plates and lysates are transferred into the filter plates. Filter-collection plate assemblies are spun in a centrifuge at 980 g for 5 minutes. Created with BioRender.com

FIG. 4. Re-arraying of lysates. After depth filtration, lysates are transferred to 4 new 24-well deep well plates as shown above. Created with BioRender.com

FIG. 5. Deck layout description. This figure shows a Hamilton Star deck layout for mid-scale protein purification using IMCS tips. The deck holds a trough for removal of storage buffer from IMCS tips, four racks of 1 mL Hamilton filtered tips for transferring buffers into plates, one rack of 1 mL IMCS tips, four troughs for purification buffers, two blotting stations, ten 24-well plates and three 96-well plates used in this method. This purification method has the capacity to process 24 unique sample of up to 24 mL volume, using up to ninety-six 1 mL IMCS tips. 24-well sample plates allow for up to four 1 mL IMCS tips to be used simultaneously per well, which increases the amount of resin used to purify each sample. Three wash stations with three associated waste plates are incorporated to provide clean wash reagent for each wash cycle prior to elution. Blotting stations are implemented prior to the wash and elution steps to remove residual droplets on the IMCS tips.

FIG. 6. Elution plate layout. Eluates are pooled together into a single well from 4 adjacent wells after affinity purification prior to further characterization. Created with BioRender.com

FIG. 7. User Interface Description. This custom user interface allows for an efficient and accurate protein purification process. The modular selection of each step within the “Purify” box allows for run recovery and maximum user customization run to run if required. The “Sample” selection allows the scientist to vary the number of sample binding plates run to run. The “Reagent” selection allows for exclusion of reagent addition. This aids in buffer optimization and screening for development runs. The “Exceptions” section allows scientists to perform additional steps including excess storage buffer removal from Integrated Micro Chromatography Systems (IMCS) tips into a trough filled with water prior to the purification run. This supports improved liquid handling so that no carry over storage buffer negatively affects the mixing cycles during the protein purification process.

FIG. 8. Schematic of flow path for Thermo Fisher Vanquish™ Duo HPLC system. This is a stacked system with dual gradient pump, dual split autosampler, column compartment, Variable wavelength detector (VWD) left and right detectors. Fraction collector allows fraction collection for up to 4×96 well plates. Two separate fraction collectors are used for left and right systems. Created with BioRender.com

FIG. 9. Vanquish Chromeleon™ 7 settings. This screen shows the UV left module on the Vanq Left system. Lamps are turned on with a toggle switch under UV and Vis Lamp. Wavelength is selected as 280 nm for SEC purifications run. Data collection rate of 10.0 Hz and response time of 0.5 sec are chosen as default settings for all purification runs.

FIG. 10. Vanquish Chromeleon™ 7 method settings. The Chromeleon™ console screen shows the method to create “sequence” in “Data” tab for samples to run on Vanquish left arm. Location in autosampler, volume of sample to be injected in the column and Instrument method are selected in the sequence for each sample. Instrument method is created and parameters for each module are selected. The method can be used for all purification runs if same column is used.

FIG. 11. Mid-scale purification data for multi-protein complex. SDS-PAGE gel analysis and size exclusion chromatography chromatogram data for multi-protein complex purified from insect cells. Protein complex was purified from Sf9 cells using the affinity purification protocol described for FLAG tagged proteins. Eluates were concentrated and further cleaned using SEC at wavelength 280 nm. Fractions corresponding to peaks were further analyzed on gel. Representative chromatogram and analyzed fractions is shown for one of the sample loaded in lane 6 of first gel.

FIG. 12 shows a size exclusion chromatography (SEC) chromatogram analysis of 8 protein multi-protein complex purified from conventional batch mode of large scale purification. Column used was Superdex® 200 increase 10/300 and buffer used was 20 mM HEPES pH 7.5, 150 mM NaCl, 1 mM DTT. Protein was purified from 3 L culture of Sf9 cells using affinity M2-ant FLAG resin, ion exchange, concentrated and loaded onto SEC for final purification step.

### DETAILED DESCRIPTION OF CERTAIN EMBODIMENTS

### 1. Definitions

Unless otherwise defined, scientific and technical terms used in connection with the present invention shall have the meanings that are commonly understood by those of ordinary skill in the art.

In this application, the use of “or” means “and/or” unless stated otherwise. In the context of a multiple dependent claim, the use of “or” refers back to more than one preceding independent or dependent claim in the alternative only. In this application, the article “a” or “the” preceding an item generally means “one or more” of such an item, unless context dictates that only one such item can be present. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

“Affinity chromatography” refers to a method of separation based on a specific interaction between molecules of an affinity column and a particular polypeptide to be purified, such as the binding of the polypeptide to a ligand, the binding of a His-tag or other peptide tag to a metal ion or to a specific antibody or the like placed a chromatography matrix.

“Size exclusion chromatography (SEC)” is a chromatographic method of separating molecules by size.

The term “matrix” is used herein to refer to a chromatography material, such as an affinity chromatography material or size exclusion chromatography (SEC) material. In some embodiments, the matrix may comprise beads or particles comprising a material to which a polypeptide may selectively bind, such as comprising a chelating ligand bound to nickel. In some embodiments, a matrix of affinity chromatography material may be placed in column through which the material to be purified may flow. In other cases, it may be placed in a spin column, or placed on a plate or chip or other device. In some cases, the matrix could comprise beads or particles, such as magnetic particles, that can be separated from a solution, e.g., by introduction of a magnet.

An “eluate” as used herein refers to material that has been eluted from a chromatography matrix or column by application of an elution buffer.

The terms “polypeptide” and “protein” are used interchangeably and refer to a polymer of amino acid residues. Such polymers of amino acid residues may contain natural and/or non-natural amino acid residues, and include, but are not limited to, peptides, oligopeptides, dimers, trimers, and multimers of amino acid residues. The terms also include polymers of amino acids that have modifications such as, for example, glycosylation, sialylation, and the like, or that are complexed with other molecules.

The term “isolated” or “purified” polypeptide or protein means a polypeptide that has been at least partially separated from one or more contaminants. In some embodiments, a polypeptide is purified to greater than 80%, 90%, 95%, or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of protein and antibody purity, see, e.g., Flatman et al., J. Chromatogr. B 848:79-87 (2007).

As used herein, an “automated” or “automatically controlled” process is one that is capable of being run, for example, by a computerized control system with appropriate software, as opposed to a system that requires an active, manual intervention during or between at least one step, such as to move an analyte-containing sample from one part of the system to another. In some embodiments, the process is automated by software that controls the movements or positions of one or more pumps, valves, and/or tees during the course of the process, which movements or positions, in turn, control the flow of buffers and eluates through the system.

The term “sample” herein refers to an amount or volume or portion of a protein purification product or intermediate, such as a portion of a cell culture or a portion of a cell lysate or supernatant from a cell lysate, for example. The amount or volume or portion can be up to 100% of the product or intermediate. Accordingly, a cell culture “sample,” for instance, refers to an amount or volume or portion of a cell culture that is generated in or used by a polypeptide purification method, such as those described herein. In some cases, each cell culture sample is grown up in one single container, such that, in some embodiments, multiple such samples are subjected to methods herein in parallel for purification of polypeptides expressed by the cells.

A “multi-well plate” or similar terms, such as a 24-well plate, or 96-well plate, for example, refer to a plate comprising a number of individual wells into which liquid may be dispensed.

A “pipette tip” as used herein, for example, for housing an affinity matrix, generally refers to a tube that may be used for dispensing liquid. In some cases, the top of the pipette tip is capable of attaching to a pipette instrument (e.g., a multi-pipettor) for use in transferring liquid into or out of the tip.

The term “plurality” herein refers to two or more. In some embodiments, a plurality may also comprise more than two, such as from 4-96, or rages within those numbers.

As used herein, a method step that is performed “in parallel,” for example, on a plurality of samples means that the method step is performed on the plurality of samples at the same time. In some cases, for example, automated equipment can be used to perform methods on several samples at the same time. In other cases, automated equipment is not necessary. For example, one means of processing samples in parallel involves using liquid dispensing or collecting equipment that dispenses or collects liquid from multiple samples at one time or using one instrument, such as a multi-pipettor, which could be operated manually or automatically depending on the circumstances.

### 2. Methods

This disclosure relates, for example, to methods and systems for purifying a polypeptide. In some embodiments, for example, the method starts with growth of a plurality of appropriate cell culture samples at “mid-scale” such as at a scale of 20-500 mL. In some embodiments, the cell culture samples are grown at a scale of 30-250 mL, 50-250 mL, 30-200 mL, 50-200 mL, or 100-200 mL, for example. Methods herein are compatible with insect cell culture, mammalian cell culture, and bacterial cell culture. The type of cell culture may be chosen based on the polypeptides intended for purification, for example. Accordingly, methods herein, in some embodiments, comprise growing a plurality of mammalian, insect, and/or bacterial cell culture samples or alternatively, purifying cell lysate samples that are obtained from a plurality of mammalian, insect, and/or bacterial cell culture samples. Thus, in some embodiments, the methods comprise growing insect cell culture samples for expressing a polypeptide, or starting with cell lysates obtained from such samples. In some embodiments, the methods comprise growing mammalian cell culture samples for expressing a polypeptide, or starting with cell lysates obtained from such samples. In some embodiments, the methods comprise growing bacterial cell culture samples for expressing a polypeptide, or starting with cell lysates obtained from such samples. In cases where the same type of cell culture sample is used (e.g., the plurality of cell culture samples are all from insect cell culture), depending on the conditions, the different cell culture samples may in some embodiments also be grown in parallel for greater efficiency. In cases where different cell culture conditions are compared for example, such as mammalian vs. insect cell cultures, the cell culture samples might be grown at different times and under different conditions due to the different needs of each host cell type. In some embodiments, the cells are insect cells, such as Sf9 or T.ni cells. In some embodiments, the cells are mammalian cells, such as HEK293 or CHO cells.

Methods herein are useful for purifying polypeptides from a plurality of mid-scale cell culture volume samples. For example, one may grow up multiple different cell cultures, each comprising host cells for expressing polypeptides, and then purify each of the cell cultures largely in parallel, in order to perform a mid-scale polypeptide purification on multiple samples. For example, in this way methods herein may be useful in, for instance, comparing different cell culture conditions for production of the same polypeptide. They may also be useful in, for instance, rapid, mid-scale production of multiple different polypeptides in parallel, such as variants of a polypeptide with different mutations or the like, so that all of the different polypeptides may be tested and compared in a downstream assay. Unlike small scale protein production methods, which may be on the order of 1-5 mL of cell culture, a mid-scale cell culture, as here may also better reflect the conditions of an eventual larger scale cell culture of 1 liter or more that may be used in later commercial production. And methods herein allow for highly pure protein to be produced at mid scale, and, as noted above, parallel testing of many different cell culture conditions to assist in determining the best conditions for larger scale production. Furthermore, in some embodiments mid-scale production methods herein allow for production of, for example 24-48 different protein purifications per week, allowing for high throughput analysis of multiple protein samples.

Methods herein also allow for cell culture samples to be grown at mid-scale, as noted above, and to be both grown and lysed in the same container, such as a flask or the like. In some embodiments, cells are grown and lysed in the same container, such as a flask. In other embodiments, cells are grown in one container but lysed in a different container. In some embodiments, the cells are lysed using glass beads and shaking. In some such cases, the cells are lysed by adding glass beads to the container in which the cells were grown, thus growing and lysing the cells in the same container. In some embodiments, the cells are not lysed with sonication. For example, glass beads may be added to the cell culture solution at the end of the growth phase, and the containers may be agitated in a shaking incubator or with similar equipment in order to allow lysis of the cells. Exemplary protocols for growth and lysis of cells are provided in the Examples herein.

Once cells are lysed, the cell lysis solution (e.g., the lysate) may be centrifuged, either in the same container as used for growth and lysis, or in a different container, in order to separate cellular debris from the supernatant comprising the expressed polypeptide or polypeptides. Accordingly, a plurality of grown, lysed, and centrifuged cell culture samples, each at mid-scale, yields a plurality of supernatant samples following centrifugation. In some embodiments, the centrifugation may be performed on the plurality of samples in parallel, such as by placing them into a centrifuge in the same run or in subsequent runs performed on the same day or roughly the same time. These samples, in some embodiments, may then be clarified, such as by depth filtration or surface filtration, in order to further remove contaminants from the expressed polypeptides. In some embodiments, clarification is by depth filtration. This then yields a plurality of clarified supernatant samples that can be placed into wells of a multi-well plate and further purified, for example, by chromatography. In some cases, the clarification may be done in parallel on multiple samples. The Examples below provide exemplary protocols for conducting clarification, for example, by depth filtration. For instance, filters may be placed over individual tubes or over wells of a multi-well plate and supernatant collected after centrifugation of cell lysates may be transferred onto the filters such that it flows through the filters and is collected into the appropriate tubes or wells below the filters. In this way, a plurality of samples can be clarified by filtration and placed into tubes or into wells of a multi-well plate. In some cases, particularly for larger scale cell cultures, the volume of supernatant is large enough that one cell culture sample will be split among several wells of a multi-well plate or among several tubes. In some cases, while supernatant from a single cell culture may be split among several tubes or wells of a plate, supernatants from different cell culture samples are not combined into the same well or tube. Instead, they are processed in separate tubes or wells, for example, in parallel, so that the conditions can be directly compared.

The clarified supernatant may be collected from the clarification step, such as after flowing through a depth filter or other filter into wells of a multi-well plate or into separate tubes. In this way, the process of cell culture, lysis, centrifugation and clarification yields a plurality of clarified supernatant samples that are placed into wells of a multi-well plate or into a set of tubes for further processing. In some embodiments, the volume of clarified supernatant that is placed into each well or tube is 2-30 mL per well or per tube. In some cases, the volume is 2-10 mL, 4-10 mL, 4-8 mL, 10-30 mL, 10-20 mL, 5-15 mL, 15-30 mL or 20-30 mL. In some cases, the clarified supernatant samples have been placed into a multi-well plate.

In some cases, cell lysate samples or clarified supernatant samples are next subjected to affinity chromatography in order to purify the polypeptide or polypeptides that the cells were intended to express for purification. In some cases, the polypeptides are tagged with an appropriate tag that is recognized by an affinity reagent such as an antibody or hapten located in the affinity matrix used for the chromatography. Nonlimiting examples of such tags include a polyhistidine, FLAG, streptavidin, glutathione-S-transferase (GST), or maltose binding protein (MBP) tag. An affinity matrix may be selected that specifically recognizes the chosen tag. In some cases, if the cells express more than one protein, one or more than one of the proteins may be designed to express the tag, so that the affinity matrix will retain the tagged proteins or protein complexes that comprise the tagged proteins. In some embodiments, the affinity chromatography matrix is present in a pipette tip, so that the transfer of liquid into and out of the matrix is simplified, and so that the affinity matrix can be used in conjunction with a multi-pipettor instrument and optionally so that it can be automated. For example, if samples are placed into a multi-well plate or tubes arranged in an appropriate pattern, a multi-pipettor can be used to draw up the liquid in the wells or tubes and place it into a pipette tip comprising the affinity matrix. In some embodiments, the affinity matrix in the pipette tip can subsequently be washed to remove unbound proteins. In other embodiments, a wash step is not performed. The bound proteins on the matrix can then be eluted. The eluate in some cases can be placed into a new multi-well plate or set of tubes, or in some instances, into the original multi-well plate or set of tubes, either for analysis or for subsequent processing. In some cases, the pipette tip comprising the affinity matrix has a volume of 0.5 to 2 mL, such as from 1 to 2 mL, or 0.5 to 1.5 mL, or 0.5 mL, or 1 mL, or 1.5 mL, or 2 mL. In some cases, the affinity matrix in the pipette tip has a bed volume of from 30 to 100 μL, such as from 30 to 70 μL, or from 40 to 50 μL, or 30 μL, 40 μL, 50 μL, 60 μL, 70 μL, or 100 μL. In some cases, the affinity chromatography step is automated, such that the liquid is added and removed to the appropriate wells, pipette tips, and tubes, as needed, in an automated fashion using appropriate equipment and software to direct a multi-pipettor or similar device accordingly. In other cases, this step is performed manually, but nonetheless is performed on the samples in parallel, such as with a multi-pipettor.

In some cases, further purification steps are performed after the affinity chromatography, as needed, depending upon the protein to be purified. For example, in some cases, size exclusion chromatography (SEC) may be performed following affinity chromatography. In some cases, the SEC chromatography is performed on an SEC matrix comprising particles with pore size between 140 and 500 Angstroms, and/or with a particle size of 3 to 5 microns, and/or with a molecular weight range of 5 to 700 kDa. The protein may then be obtained from SEC fractions corresponding to the appropriately sized polypeptide or polypeptide complex, which may be monitored, for example at 280 nanometer wavelength. Depending on the available equipment, in some cases, SEC may be performed on the affinity chromatography eluate from each cell culture sample sequentially, or in other cases, it may be performed in parallel, and in some cases, the associated liquid dispensing and fraction collection may be automated.

Accordingly, methods for purifying one or more polypeptides herein may begin with growth of cell culture samples to express the polypeptides for purification, or from a subsequent step such as the affinity chromatography of a plurality of cell lysate samples, such as after centrifugation and clarification. Exemplary methods herein include a method for purifying one or more polypeptides from a plurality of mammalian cell culture samples, insect cell culture samples, or bacterial cell culture samples, comprising:
(a) growing a plurality of mammalian, insect, and/or bacterial cell culture samples expressing one or more polypeptides for purification at a scale of 20-500 mL;
(b) lysing the cells of the cell culture samples, optionally wherein parts (a) and (b) for each of the plurality of cell culture samples are conducted in the same container;
(c) centrifuging the lysed cell culture samples and collecting a plurality of supernatant samples from the cell culture samples;
(d) clarifying the plurality of supernatant samples by filtration, such as depth filtration;
(e) placing the plurality of clarified supernatant samples into wells of a multi-well plate, wherein the supernatant samples have a volume of 2-30 mL per well; and
(f) subjecting the plurality of supernatant samples in the wells of the multi-well plate to affinity chromatography conducted with an affinity matrix in a pipette tip and placing the eluate from the chromatography into wells of a multi-well plate; wherein part (f) or parts (e) and (f) are conducted on the plurality of samples in parallel. In some cases, part (f), or parts (e) and (f) are automated, or parts (d) to (f) are automated. In some cases, only part (f), or parts (e) and (f), or parts (d) to (f) are conducted in parallel (i.e., on multiple samples at the same time). In some cases, all of the parts of the method are performed in parallel. In some cases, the multi-well plate could be replaced by an array of appropriate tubes for holding the liquid samples.

Additional examples include a method for purifying one or more polypeptides by affinity chromatography starting from a plurality of clarified cell lysis supernatant samples in a multi-well plate, which were obtained from a plurality of cell culture samples by a process comprising:
(a) growing a plurality of mammalian, insect, and/or bacterial cell culture samples expressing one or more polypeptides for purification at a scale of 20-500 mL,
(b) lysing the cells of the cell culture samples, optionally wherein parts (a) and (b) for each of the plurality of cell culture samples are conducted in the same container;
(c) centrifuging the lysed cell culture samples and collecting a plurality of supernatant samples from the cell culture samples;
(d) clarifying the plurality of supernatant samples by depth filtration; and
(e) placing the plurality of clarified supernatant samples into wells of the multi-well plate, wherein the supernatant samples have a volume of 2-30 mL per well;
wherein the method comprises:
(f) subjecting the supernatant samples obtained from the process of (a)-(e) to affinity chromatography conducted with an affinity matrix in a pipette tip and placing the eluate from the chromatography into wells of a multi-well plate; wherein, the method is conducted on the plurality of clarified supernatant samples in parallel, and optionally wherein the method is automated. Thus, for example, the method may begin with a sample ready for affinity chromatography, which was previously prepared following parts (a)-(e) above. In some cases, part (f), or parts (e) and (f) are automated, or parts (d) to (f) are automated. In some cases, only part (f), or parts (e) and (f), or parts (d) to (f) are conducted in parallel (i.e., on multiple samples at the same time). In some cases, all of the parts of the method are performed in parallel. In some cases, the multi-well plate could be replaced by an array of appropriate tubes for holding the liquid samples.

A further exemplary method herein includes a method for affinity purification of polypeptides from a plurality of cell culture supernatant samples expressing one or more polypeptides for purification, comprising: obtaining a multi-well plate comprising a plurality of clarified cell culture supernatant samples in wells of the multi-well plate at a volume of 2-30 mL per well, and subjecting the plurality of clarified cell culture supernatant samples to affinity chromatography conducted with an affinity matrix in a pipette tip and placing the eluate from the chromatography into wells of a second multi-well plate; wherein, the method is conducted on the plurality of supernatant samples in parallel, optionally wherein the method is automated. In some embodiments, the plurality of cell culture supernatant samples were obtained from mammalian, insect, and/or bacterial cell culture samples expressing one or more polypeptides, which were grown at a scale of 20-500 mL. In some embodiments, the cell culture samples were subjected to one or more of lysis, centrifugation, and clarification prior to use in the method, optionally wherein growth and lysis of the cells (if lysed) were conducted in the same container. For example, in some cases, the clarified cell culture supernatant samples comprise polypeptides that are secreted by the cells, in which case it may not be necessary to lyse the cells, and instead, the cell culture, after sufficient growth, may simply be centrifuged and/or clarified prior to the affinity chromatography.

In any of those examples, the method further may comprise, if desired:
(g) conducting size exclusion chromatography (SEC) on the eluate of the affinity chromatography; and
(h) fractionating polypeptides from the SEC into wells of a multi-well plate. In some options, parts (g) and (h) are automated.

In any of these methods, a new multi-well plate may be used in each step. In other cases, sample may be obtained from a multi-well plate, processed, and returned to the same plate. In any of these methods, in some embodiments, a clarified supernatant sample corresponding to a single cell culture sample is placed into more than one well of the multi-well plate. In some cases, clarified supernatant samples from different cell culture samples are placed into different wells of the multi-well plate. In some cases, from 8 to 96 clarified supernatant samples are processed in parallel, such as from 8 to 48, from 8 to 24, from 12 to 48, or from 12 to 24. The number of samples that can be processed in parallel during affinity chromatography, for example, may depend on the number of wells in a compatible multi-well plate and correspondingly on the number of pipettors in a corresponding multi-pipettor for transferring liquids. In some cases, a 24 well plate is chosen, such that up to 24 samples can be processed in parallel, i.e., 2-24, 8-24, 2-12, or 12-24, etc. In other cases, a 48 or 96 well plate may be used. Alternatively, an array of tubes may be used in lieu of a plate.

In some methods above, cells are lysed by addition of glass beads with shaking and/or wherein the cells are not lysed by sonication. In some cases, the plurality of cell culture samples are grown at a scale of 30-250 mL, 50-250 mL, 30-200 mL, 50-200 mL, or 100-200 mL. In some cases, the pipette tip comprising the affinity matrix has a volume of 0.5 to 2 mL, such as from 1 to 2 mL, or 0.5 to 1.5 mL, or 0.5 mL, or 1 mL, or 1.5 mL, or 2 mL. In some cases, the affinity matrix in the pipette tip has a bed volume of from 30 to 100 μL, such as from 30 to 70 μL, or from 40 to 50 μL, or 30 μL, 40 μL, 50 μL, 60 μL, 70 μL, or 100 μL. In some cases, the polypeptides are tagged with a polyhistidine, FLAG, streptavidin, glutathione-S-transferase (GST), or maltose binding protein (MBP) tag, and wherein the affinity matrix recognizes the tag. In some cases, the method comprises parts (g) and (h), and wherein the SEC chromatography is performed on an SEC matrix comprising particles with pore size between 140 and 500 Angstroms, and/or with a particle size of 3 to 5 microns, and/or with a molecular weight range of 5 to 700 kDa.

In some cases, methods herein further comprise performing structural or functional analysis on the purified polypeptides, such as cryoelectron microscopy, mass spectrometry, protein-protein interaction assays such as surface plasmon resonance, or homogeneous time resolved fluorescence assays.

In some cases, the polypeptides expressed from the cell culture samples comprise recombinant protein complexes. In some cases, the polypeptides do not comprise antibodies or antibody subunits. In some cases in methods herein, the cell culture samples are insect cell culture samples, such as Sf9 or T.ni cell culture samples. In some cases, the cell culture samples are mammalian cell culture samples, such as HEK293 or CHO cells, among other examples. In some cases, a mix of different cell culture types may be used, for example, to compare insect cell vs. mammalian cell protein production and the like. In other cases, the methods may be used to compare the same host cell type grown under different media or conditions, for example. In other cases, the same cell type optionally with the same growth and media conditions is used, but the polypeptides vary, such as to compare various polypeptide mutants against each other.

### 3. Systems and Kits

The present disclosure also relates to systems capable of conducting methods described herein, including those described in the section above, and kits comprising reagents or components useful in performing the methods herein. In some embodiments, systems may include a device capable of automating affinity chromatography in pipette tips. In some embodiments, a system may also comprise a device capable of transferring a plurality of cell lysate samples, such as after centrifugation and clarification, to and from an array of tubes or wells of a multi-well plate, and optionally further capable of conducting affinity chromatography on the plurality of samples in the tubes or wells of the plate, for example, by operation of a multi-pipettor or similar component used to transfer liquid in and out of tubes or wells of multi-well plates.

The disclosure herein also encompasses kits comprising reagents for performing methods herein, such as reagents for affinity chromatography, i.e., pipette tips comprising affinity matrix, buffers for performing affinity chromatography (including wash, equilibration, and/or elution buffers), control samples, and the like. Kits may also comprise appropriate plates or tubes for holding samples. Kits may further comprise directions for use.

### EXAMPLES

### Example 1: Parallel Protein Expression of 24 Samples from Insect Cell Culture

Insect cells are widely used as a heterologous expression system to produce high levels of eukaryotic recombinant proteins with simple post-translational modifications such as phosphorylation and glycosylation (Shi & Jarvis, 2007) (Jarvis, 2009). Insect cells have the cellular machinery to fold and target these proteins to the correct localization, and provide soluble, non-aggregated proteins in significant quantities. This system is proven to be particularly useful for expressing any class of macromolecular assemblies, including histone methyltransferases, DNA repair enzymes, kinetochores, ubiquitin ligases, and viral capsid complexes (Abdulrahman et al., 2015; Osz-Papai et al., 2015).

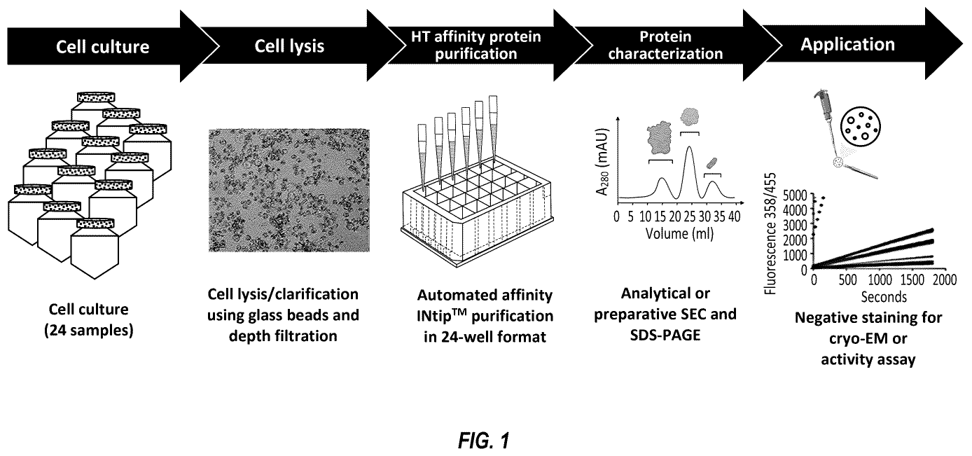

This Example focuses on insect cell expression and describes a mid-scale protocol used to perform successful expression and purification screens of intracellular multiprotein complexes. While protein co-expression using single Open Reading Frame (ORF) encoding plasmids is widely used in baculovirus-mediated insect cell expression, the use of multi-ORF or polycistronic plasmid vectors offers a significant advantage for reconstituting protein complexes with two or more proteins, since this approach uses fewer viruses during co-expression and also ensures uniform expression in insect cells (Snead, Wall, Ambrose, Esposito, & Drew, 2022). Overexpression of multiple proteins from a single plasmid vector can be achieved by either using individual promoters to drive the transcription of each ORF or by using a polycistronic construct, in which a single mRNA transcript encodes multiple proteins. This Example also describes the design and implementation of a two-step sequential purification process that includes affinity capture and Size Exclusion Chromatography (SEC) (FIG. 1).

The process is designed to be an end-to-end solution from culture to protein purification. It enables the expression and semi-automated purification of 24 intracellular proteins or complexes in parallel from insect cells at 200 mL scale using affinity purification on a Hamilton STAR™ liquid handler followed by SEC on a Thermo Fisher Vanquish™ Duo system. Affinity chromatography using INtip™ technology by Integrated Micro Chromatography Systems (IMCS) was performed in a high-throughput manner (Kates et al., 2023) (Kates, Tomashek, Miles, & Lee, 2020). The protocol described in this Example used the dispersive micro-extraction INtip™ platform that leverages turbulent mixing of the resin within a pipette tip to increase interaction time between resin and sample. This was coupled with an automated liquid handling system—Hamilton® Microlab STAR™ workstation—to enable parallel rapid purification of recombinant proteins in a consistent and high-throughput manner.

SEC was then performed on a Thermo Fisher Vanquish™ Duo system coupled with an autosampler that injects samples sequentially and allows two simultaneous purifications using separate buffer lines with a Horizon Dual pump, separate injection loops, and parallel columns and detectors. This method allowed high-quality protein complexes to be purified, which were further evaluated for downstream applications and assays to identify refined and optimized solutions for large scale production and purification. The following more specific protocols, related to specific steps in the platform, are provided below.

Basic Protocol 1 describes the methodology that was used to generate P1 baculovirus in Sf9 cells using co-transfection of linearized baculovirus bacmid BestBac™ with the gene of interest, subcloned into a baculovirus transfer vector, such as pAcGP67. This protocol also describes how to amplify the baculovirus to generate P2 and P3 stocks in Sf9 cells.

Basic Protocol 2 describes infection of Sf9 and T.ni cells at 200 mL scale with P3 virus, that was used to generate biomass expressing protein(s) of interest. Also discussed here is a co-expression approach through co-infection of cells with multiple viruses.

Basic protocol 3 describes cell lysis and the preparation steps for INtip™ affinity chromatography purification that were performed on Hamilton STAR™ workstation.

Basic protocol 4 describes the SEC method that was developed on the Thermo Fisher Vanquish™ system to provide analytical and preparative purification at mid-scale.

Support Protocol 1 describes a Glycoprotein 64 (GP64) staining assay that was used to assess the quality of baculovirus.

Support Protocol 2 describes the automated method and steps of mid-scale INtip™ affinity purification that were performed on the Hamilton STAR™ workstation.

Support Protocol 3 describes the detailed method that was built using Chromeleon™ software to support the SEC component of the mid-scale platform.

### Basic Protocol 1

### Baculovirus Generation Via Homologous Recombination

This protocol assumes that the initial step of cloning the gene(s) of interest has been accomplished. DNA encoding the genes of interest was cloned into modified versions of the commercially available baculovirus transfer vector pAcGP67 (BD Biosciences, Brøndby, Denmark), adapted for high-throughput cloning. Recombinant genes were expressed as fusion proteins when cloned into one of the available restriction enzyme sites under the control of the strong baculovirus polyhedrin promoter. The expression cassette was flanked by segments of the baculovirus genome to facilitate transfer into the linearized baculovirus DNA by homologous recombination in insect cells (Murphy & Piwnica-Worms, 2001). The GP67 secretion signal sequence in pAcGP67 vector was removed for cloning of intracellular proteins and retained for cloning of secreted proteins. The same expression vector backbone was also used for generating polycistronic or multi-ORFs constructs.

The strategies used to generate constructs containing two or more genes, such as the use of Internal Ribosome Entry Site (IRES) and self-cleaving 2A peptides, are widely used approaches for generating polycistronic constructs, since both strategies simultaneously express two or more separate proteins from the same mRNA under the control of a single promoter. Multi-ORF constructs can be also rapidly generated from pAcGP67 vector by subcloning genes of interest with individual promoters, such that the mRNA transcript of each target gene can be independently generated for efficient overexpression of protein complexes. When higher order protein complexes need to be achieved, the number of promoter systems can be extended which requires modification of the pAcGP67 vector.

Promoters varying in transcriptional strength can be used to achieve variable multigene expression stoichiometry. For insect cells, early or late promoters can be used to control the expression of different genes in the infection cycle. Multigene co-expression is context-specific and different promoters can result in variable multigene expression stoichiometry.

In this Example, baculovirus was generated in insect cells by means of homologous recombination (Kitts, Ayres, & Possee, 1990; Kitts & Possee, 1993). The protocol herein provides the steps of co-transfecting the linearized bacmid with the expression transfer vector pAcGP67 encoding the gene(s) of interest. The co-transfection resulted in the generation of recombinant baculovirus, which was then amplified to a higher titer (1×10⁸-1×10⁹ pfu/mL) so that it could be used for recombinant protein expression. This protocol was optimized for high throughput workflows and can accommodate the generation of up to 96 recombinant baculovirus samples at a time.

### Materials

### Materials for the First Section are Listed Below:

Recombinant Transfer Vector pAcGP67 or similar, expressing gene(s) of interest at 40 ng/μL concentration (See Background Information/Basic Protocol 1)
Empty transfer vector with matching backbone to the vector used for virus generation to be used as a control, at 40 ng/μL concentration
BestBac™ 2.0 Linearized Bacmid-Stock concentration 0.1 mg/mL (Expression Systems #91-002)
TransIT™ Insect Transfection Reagent (Mirus #MIR6100)
Cells from Sf9 cell line in logarithmic phase of growth and >95% viability between 2×10⁶ and 7×10⁶ cells/mL in ESF921 media (Expression Systems #94-001F)
ESF921 Insect Cell Media heated to 27° C. (Expression Systems #96-001-01)
Heat Inactivated FBS (Thermo Fisher #16140-071)
96-well deep-well plates, sterile (Axygen #p-2 mL-sq-c-s)
96-well round bottom deep-well plates, sterile (Thompson #93113-S)
24-well Axygen deep-well round bottom plate, sterile
15 mL conical tubes
AeraSeal™ (Millipore Sigma #A9224-50EA)
AlumaSeal™ (T790080-5)
96-well Duetz sandwich cover with 0.8 mm holes (Kuhner Shaker Inc. #104118)
Sterile pipette tips (p20, p200, p1000)
   Matrix Tubes 1.4 mL (Cat #3712-11)
U-Bottom Assay Plates (Falcon #353910)
Zymo Clean N′ Concentrate Kit (Zymo #D4033)
Sterile Reagent Reservoirs
70% EtOH for hood disinfection
150 mL sterile media bottle

### Second Section Materials:

Pipettes of choice, preferably 8-channel (p20, p200, p1000)
Orbital Shaker, 3 mm throw set at 1000 rpm at 27° C.
Tabletop centrifuge (to spin down assay plate and DNA plate)
Sterile Serological Pipettes
Laminar flow hood (Labconco Purifier BSC Class II, or equivalent
   Protocol Steps with Step Annotations:

### Generating P1:

In a sterile laminar flow hood, Master Mixes #1 and #2 were prepared in sterile 15 mL conical tubes. The amount of master mix to be generated depends on the number of recombinant transfer vector DNA constructs to be expressed. At least 10% extra should be prepared to ensure sufficient volume for each sample. An empty vector pAcGP67 was used as a positive control along with BestBac™ linearized bacmid DNA. Untransfected cells were be used as negative control.

| Master Mix 1: Diluted | Master Mix 2: Diluted |
|---|---|
| BestBac 2.0 -- Per Sample | TransIT - Per Sample |
| 25 μL ESF921 Media | 25 μL ESF921 Media |
| 0.5 μL BestBac 2.0 Linearized | 0.5 μL TransIT Insect |
| Bacmid DNA (Stock 0.1 mg/mL) | Transfection Reagent |

2.5 μL of transfer vector DNA was added at 40 ng/μl concentration for a total of 100 ng into corresponding wells in the 96 well, deep-well Axygen plate. For larger DNA inserts (>5 kb) it is recommended to double the amount of DNA (200 ng)

The DNA plate was spun down at 1000 g for 1 minute to ensure no air was trapped in the liquid. 25 μL Master Mix 1 was added into each well and gently pipetted 3 times to mix. 25 μL Master Mix 2 was added into each well and gently pipetted 3 times to mix. The plate was sealed with an aluminum seal and incubated for 20 minutes at room temperature to allow complexes to form. Keep plate in sterile laminar flow hood during incubation.

A Sf9 cell suspension was prepared at density of 1.0×10⁶ cells/mL in ESF921 media. The volume is dependent on the number of samples, with 550 μL used per sample. 550 μL cell suspension was added per well for a total volume of 602.5 μL per well. The plate was sealed with an AeraSeal™ and covered with a Duetz™ sandwich cover. The plate was shaken at 1000 rpm on 3 mm diameter orbital shaker at 27° C. for 6 days. Fabric seals, like AeraSeal™, allow for maximal culture aeration. Avoid plastic porous seals as they have been shown to limit oxygen intake in our hands. Duetz™ sandwich covers reduce the amount of evaporation during shaking incubation. Evaporation can have a negative effect on transfection efficiency. 6 days incubation for P1 was a necessary amount of time for the recombinant virus to gain titer and it is not recommended to shorten this incubation time to less than 5 days.

### Generating P2 & P3:

On day 6, 10 μL of P1 cell suspension was transferred into 1000 μL of fresh Sf9 cells at 1.0×10⁶ cells/mL in a sterile 96-well Thompson deep-well plate. 1010 μL is about the maximum volume per well possible to maintain sufficient aeration and avoid splashing on the fabric seal cover. The plate was covered with an AeraSeal™ and Duetz™ sandwich cover. The plate was incubated in a 27° C. 3 mm diameter shaker at 1000 rpm for 4 days. At the end of the 4 day incubation period, the viral titer should reach 1×10⁸-1×10⁹ pfu/mL.

The P1 plate was stored sealed with AlumaSeal at 4° C. so it could be used again for P2 amplification in case of unforeseen circumstances, such as contamination, incubator failure, etc. On day 4 of P2 incubation, cells were stained with GP64 antibody to assess virus production (refer to Support Protocol 1). A viral plaque assay can be conducted instead of a GP64 assay if no flow cytometer is available. The advantage of a GP64 assay is a faster readout—within a day—compared to 5-7 days it takes to conduct a viral plaque assay.

P2 Virus can be stored and used for future P3 generation (See Saving Virus).

P3 virus was made by transferring 5 μL of P2 cell suspension into 4 mL of fresh Sf9 cells at 1.0×10⁶ cells/mL in a sterile 24-well Axygen deep-well plate. Plate was covered with an AeraSeal™ and incubate in a 27° C. 12 mm shaker at 300 rpm for 4 days. Generating P3 virus scales up the volume of virus that can be used for mid-scale sized expression. P3 virus can be stored at 4° C. for up to 6 months.

### GP64 Assay (Assessing Virus Generation Efficiency)

Collect 50 μL of cell suspension in a 96-well U bottom plate and proceed with GP64 staining and measurement. (See Support Protocol 1)

### Saving Virus

Plates were sealed with adhesive foil seals in the hood. Ensure a good seal by using a roller. Plates were spun at 3,000×g for 10 minutes to remove cells/debris. Clarified supernatant was transferred to sterile matrix tubes. Heat inactivated FBS was added to 10% concentration for storage stabilization (i.e. ˜111 μL FBS added to 1000 μL clarified supernatant for P2, ˜444 μL FBS added to 4 mL clarified supernatant for P3). Plates were stored at 4° C. in the dark (baculovirus can degrade when exposed to light). Virus is good for up to 12 months in storage. Virus can be re-amplified from older stock before using for expression. Older viruses may have decreased titer that can affect P3 amplification. (See Table 1)

### Support Protocol 1

### GP64 Antibody Assay

This protocol utilizes Fluorescence-activated cell sorting (FACS) to verify a high titer baculovirus stock. Glycoprotein 64 is a viral protein that expresses on the membrane of successfully infected insect cells (Kitts & Green, 1999). This glycoprotein can be tagged with a phycoerythrin labeled antibody to extrapolate sufficient viral titer (Volkman & Goldsmith, 1988).

### Materials:

Materials were as follows
FACS Buffer (see Reagents and Solutions section)
7-AAD Viability Dye (Beckman Coulter #A07704)
GP64-PE Antibody (Expression Systems #97-201)
CytoFLEX™ Sheath Fluid (Beckman Coulter #B51503) or 0.2 μm filtered sqH20 (substitute) AlumaSeal™ (T790080-5)
Tabletop centrifuge (to spin down assay plate and DNA plate)
Beckman Coulter CytoFLEX™ or similar flow cytometer with “Blue” Laser (488 nm). Laser should be able to be used with Phycoerythrin and PC5.5 fluorochrome samples

### Protocol Steps:

The following describes the protocol that was used for the GP64 antibody assay.

### Sample Preparation:

Aliquot 50 μL of sample into a U bottom 96-well plate. This should be around 100,000 cells. Sample volume may be adjusted lower or higher depending on density of the culture. Cover plate with foil seal and spin at 3000 rpm for 3 minutes. Aspirate supernatant carefully to avoid touching cell pellets. Dilute PE-anti-GP64 1:125 in FACS Wash (8 μL PE-anti-GP64 per 1 mL FACS wash buffer). Resuspend pellets uniformly in 40 μL of FACS Wash with PE-anti-GP64. Cover plate with a foil seal and incubate samples at 4° C. for 20 min. Initialize CytoFLEX at this point so the system is ready for reading samples after the following steps are completed. See CytoFLEX operation manual for details. After incubation, add 150 μL of FACS wash buffer to each well. Spin at 3000 rpm for 3 minutes. Aspirate supernatant carefully to avoid touching cell pellets. Prepare appropriate volume of viability dye mix to stain for live/dead cells. Viability dye mix is 40 μL of 7-AAD per 1 mL FACS wash buffer. Resuspend pellets with 100 μL of diluted 7-AAD Dye. Incubate at room temperature for at least 5 minutes. Proceed with analysis on CytoFLEX according to step 7.

### CytoFLEX Operation

Settings on the flow cytometer: Set for 10,000 events with flow rate at “fast” mode. Flow rate and end point can be adjusted if needed. Set to PE and PC5.5 channels. Ensure the flow cytometer is set to handle U-bottom assay plates.

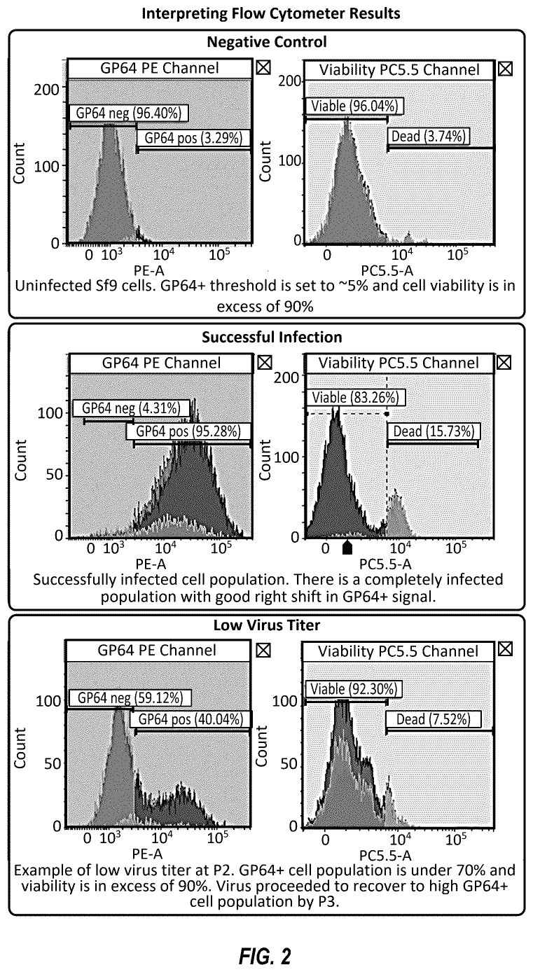

Adjust the GP64 gating and the viability gating if necessary to accommodate “GP64 positive/negative” and “viable cells/dead cells” controls (FIG. 2). This can drift slightly and it is important to adjust the gates relative to your positive (empty vector infected cells) and negative (uninfected cells) controls. Allow ˜5 minutes for the dye to enter compromised cells, bind to DNA and reach maximum fluorescence. Note: The 7AAD signal can be artificially low in the first few samples if the plate is read too soon after dye addition.

### Basic Protocol 2

### Generation of Insect Cell Biomass Expressing Target Protein(s)

This protocol was used for generation of insect cell biomass expressing the target protein. The protocol enables the generation of Sf9 or T.ni cell paste, expressing target proteins, at 200 mL scale using baculovirus generated in Basic Protocol 1.

The insect cells were infected with baculovirus and incubated for 2-3 days to allow for protein expression. The cell pellet was then harvested and frozen before cell lysis and protein extraction. Protein expression yield, recoverability, and quality may differ in Sf9 and T.ni cells, and therefore, it is advisable to test the expression in both cell lines. We have observed that the T.ni cell line often yields higher amounts of secreted, intracellular, and membrane recombinant proteins per culture volume, as compared to Sf9. However, due to a higher rate of the intracellular target protein clipping that we observe in T.ni cells, the Sf9 is often the preferred cell line for expressing intracellular proteins. The infection profile is also different in these two cell lines and may require separate optimization for optimum protein expression, for example, the starting density at infection, the Multiplicity of Infection (MOI), and the length of incubation often differ for the two cell lines when achieving optimum protein yield. While both cell lines exhibit robust culture properties, the doubling time of T.ni cells in culture is shorter compared to Sf9, which may allow to shorten the production timelines and may be an important consideration in the use of resources and reagents.

### Materials:

Materials included:
Laminar flow hood (Labconco Purifier BSC Class II, or equivalent)
Sf9 cell line in logarithmic phase of growth and >95% viability (between 2×10⁶ and 7×10⁶ cells/mL in ESF921 media, Expression Systems Cat #94-001F)
T.ni cell line in logarithmic phase of growth and >95% viability (between 2×10⁶ and 7×10⁶ cells/mL in ESF921 media, Expression System Cat #94-002F)
ESF921 insect cell growth media warmed to 27° C. (Expression Systems, cat. #96-001-01)
Sterile serological pipettes (5 mL, 50 mL) Cell counter (Beckman Coulter Vi-CELL XR or BLU or equivalent)
1 L polycarbonate Erlenmeyer flask with vent cap (Corning, cat. No. 431147)
AeraSeal (Millipore Sigma, cat. No. A9224-50EA)
500 mL conical-bottom polypropylene centrifuge tubes (Corning, cat. No. 431123)
Single-channel Pipette (1000 μL)
Sterile 1000 μL tips
Shaking incubator with 25 mm shaking diameter, maintaining 150 rpm and 27° C. (Infors or equivalent)
Rack for 500 mL tubes (Infors, cat. No. 66129)
Centrifuge with swinging rotor (Beckman or equivalent)
Polyetherimide centrifuge tube cushions for 500 mL tubes (Corning, cat. No. 431124) −80° C. freezer (Thermo Fisher or equivalent)

### Protocol Steps

Steps 1-3 of this protocol were performed in a Biosafety cabinet using aseptic technique, and are as follows:

Using a 5 mL serological pipette, aspirate 1-2 mL of Sf9 or T.ni cell stock and dispense in a sample cup of Vi-CELL counter to measure viable cell density and assess overall culture health by analyzing viability and the average viable diameter. Insect cells are maintained in ESF921 media in Erlenmeyer flasks by regular splitting every 2-3 days to a cell density of no less than 0.7×10⁶ cells/mL, while not allowing cells to reach a density over 7×10⁶ cells/mL. Culture viability should be >=95% and diameter of uninfected cells should not exceed 15.5 and 19 μm, for Sf9 and T.ni cells, respectively.

Dilute Sf9 or T.ni cells to 2×10⁶ cells/mL with ESF921 medium at 27° C. and dispense 200 mL into each 500 mL tube. To infect Sf9 cells, add 0.5 mL of P3 virus to 200 mL of cells. To infect T.ni cells, add 1 mL of P3 virus to 200 mL of cells. A guiding formula for calculating the volume of virus in milliliters (Vᵥᵢᵣᵤₛ) to be added to an insect cell culture depends on the starting cell density Dculture expressed in cells/mL, the culture volume V_{culture} expressed in milliliters, the MOI (Multiplicity of Infection) expressed in pfu/cell, and the virus titer Tᵥᵢᵣᵤₛ calculated in pfu/mL. Thus,

$${V}_{virus} = ( {D}_{culture} ) ( {V}_{culture} ) ( MOI ) / ( {T}_{virus} )$$

For a freshly generated virus with a high gp64 staining percentage, it is acceptable to assume a titer of 4×10⁸ pfu/mL. In addition, we have experimentally determined that for the majority of proteins expressed in Sf9 and T.ni cells, an MOI of 0.5 and 1, respectively, works best. Thus for Sf9 cells,

$${V}_{virus} = ( 2 \times {10}^{6} cells / mL ) ( 200 mL ) ( 0.5 pfu / cell ) / ( 4 \times {10}^{8} pfu / mL ) = 0.5 mL$$

For T.ni cells,

$${V}_{virus} = ( 2 \times {10}^{6} cells / mL ) ( 200 mL ) ( 1 pfu / cell ) / ( 4 \times {10}^{8} pfu / mL ) = 1 mL$$

For protein co-expression from different plasmids, which requires the use of multiple viruses, add equal amounts of each P3 virus to cells.

Incubate the cultures in tubes at 27° C. with shaking at 150 rpm, 25 mm shaking diameter, for 72 or 48 hours, for Sf9 and T.ni cells, respectively. Check cell viability and diameter on day 2 for T.ni and day 3 for Sf9. If cell diameter is greater than 17 or 21 μm, for Sf9 and T.ni, respectively, and viability is between 50 and 85%, proceed with harvesting. If cells diameter is less than 17 or 21 μm, for Sf9 and T.ni, respectively, please follow troubleshooting, as described in Table 1. If viability is less than 50%, please see troubleshooting, as described in Table 1.

Harvest cells by centrifugation at 2,200 g for 10 minutes. Discard the supernatant and freeze pellets at −80° C. Even if the purification is being planned on the same day, it is recommended to freeze the cell pellets for at least 10 minutes prior to cell lysis.

### Basic Protocol 3

### Mid-Scale Affinity Purification

This protocol was used to perform parallel affinity purification of 24 samples containing His- and/or FLAG-tagged proteins using IMCS tips and a Hamilton STAR liquid handler.

### Materials

Materials included:
Equilibration and Lysis Buffer (see recipe in Reagents and Solutions)
TCEP-HCl (Pierce, Cat. No. 20490)
Benzonase endonuclease (Sigma, cat. No. 101697)
Roche Complete™ EDTA-free protease inhibitor cocktail (Sigma, cat. No. 11873580001)
Glass beads (Thomas Scientific, cat. no. 1177X44 or similar)
Shaking incubator with 25 mm shaking diameter, maintaining 250 rpm and 10° C. with suitable tray to hold 500 mL tubes (Infors or equivalent)
Rack for 500 mL tubes (Infors, cat. no. 66129) 24-well depth-filtration filter plates (Orochem, Cat. No. OC24DAHL-B)
24-well plates (Axygen, cat. no. P-DW-10ML-24-C-S)
Centrifuge with swinging bucket rotor, such as Beckman JS-5.3 or equivalent
Microplate carriers for swinging buckets (Beckman Cat. No. 368905)
Support pads for microplate carriers (Beckman Cat. No. 369382)
Polyetherimide centrifuge tube cushions for 500 mL tubes (Corning, cat. No. 431124)
Reagent reservoirs (Thermo Fisher Scientific Cat. No. 95128085)
1000 μL 12-channel pipette of choice
1000 μL tips of choice
1000 μL 8-channel pipette of choice
1000 μL IMCS tips packed with 50 μL Ni-NTA resin or 50 μL M2 anti-FLAG resin (IMCS, Cat. No. DP016 or DP017, respectively)
96-well plates (Thermo Scientific, Cat. No. AB-0932)
Elution Buffer (see recipe in Reagents and Solutions)
FLAG peptide (Sigma, cat. No. MFCD01863911)
Heavy duty blotting system super absorbent pad with lint-free blotting media in an omnitray (V&P Scientific, Cat. No. VP 540 DB)
Super absorbent polypropylene pad cut to fit omnitray (V&P Scientific, Cat. No. VP 540DB1-100)
Lint-free blotting media (V&P Scientific, Cat. No. C VP 540D-100)
Hamilton Microlab STAR™
Reservoirs robotic 300 mL polypropylene for buffers (Thermo Fisher Scientific Cat. No. 12565571)
Acid Buffer (see recipe in Reagents and Solutions)
Wash Buffer (see recipe in Reagents and Solutions)
Hamilton CO-RE 1000 μL filter tips (Hamilton, Cat. No. 235940)
NanoDrop™ spectrophotometer (ThermoFisher Scientific)
NuPAGE™ sample reducing agent (10×) (Invitrogen, Cat. No. NP0009)
NuPAGE™ LDS sample buffer (4×) (Invitrogen, Cat. No. NP0007)
Thin walled 200 μL PCR 96-well plates (Axygen or similar)
Plate seals (AlumaSeal, Cat. No. T790080-5 or similar)
PCR thermocycler (Fisher Scientific or equivalent) or 95° C. water bath for boiling samples NuPAGE™ 4 to 12%, Bis-Tris, 1.0 mm, Midi Protein Gels (Invitrogen, Cat. No. WG1402BOX)
XCell4 SureLock™ Midi-Cell gel-running box (Invitrogen, Cat. no. WR0100)
NuPAGE™ MES SDS Running Buffer (20×) (Invitrogen, Cat. No. NP0002)
Precision Plus Protein™ Kaleidoscope™ Prestained Protein Standards (Bio-RAD, Cat. No. 1610375)
PowerPac™ (Bio-RAD or equivalent)
Gel knife (ThermoFisher, Cat. No. EI9010 or similar)
Trays for washing and staining gels
InstantBlue Coomassie Protein Stain (Novus Biologicals, Cat. No. ISBIL)
Lab rocking platform (VWR or equivalent)
Bio-RAD Gel Doc™ EZ Imaging System with Image Lab™ Software or equivalent

### Protocol Steps

The protocol steps used were as follows:

Keep frozen pellets on ice. All cell lysis and re-array steps should be performed on ice, as much as feasible. Leaving cell pellets, lysates, or protein samples at room temperature can result in accelerated protein degradation.

Add 24 mL of Lysis Buffer and ˜2 mL of glass beads to frozen pellets in 500 mL tubes and incubate at 10° C. with shaking at 250 rpm for 1 hr in an Infors incubator with 25 mm shaking diameter. Alternatively, one can use sonication to lyse cells but this will not allow higher throughput format. The proposed method of lysis with beads allows the incubation and lysis of 24 samples at a time.

Pre-wet 24-well Orochem filters with 1 mL of Equilibration Buffer by placing filter plates on top of 24-well collection plates and centrifuging for 2 min at 980 g. Discard flow-through and place filter plates back on top of the 24-well plates. The plate assemblies may be secured on a side with a laboratory tape through centrifugation in Step 6. Pre-wetting depth filters is essential for efficient recovery of the filtrate. One Orochem plate can be used to filter up to 4 samples. Alternatively, the lysates can be filtered using traditional PES filters, however, these filters are prone to clogging easily.

Clarify lysates by centrifugation at 2,200 g for 10 min using a Beckman floor centrifuge. Decant the supernatant into pre-labeled troughs. Discard the pellets.

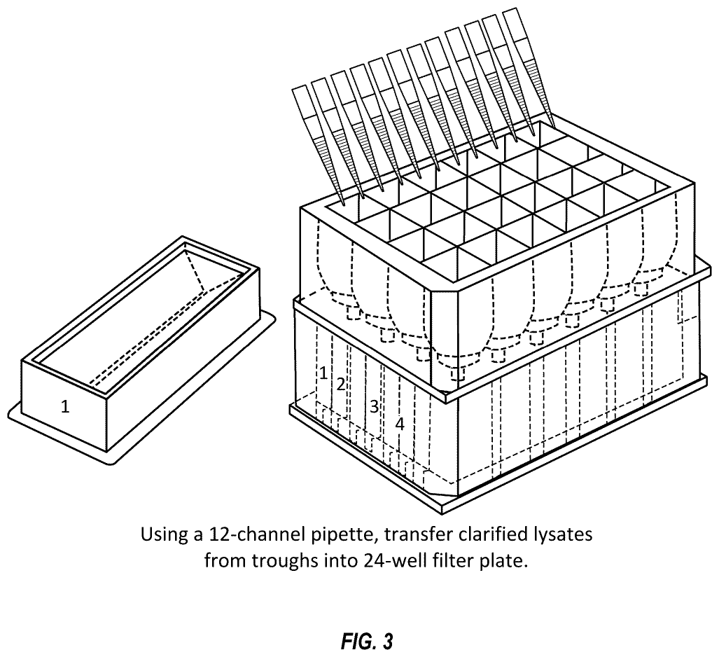

Using a 1000 μL 12-channel pipette, transfer clarified lysates from troughs, one at a time, into pre-wetted 24-well filter plates, such that each lysate is transferred into one row (6 wells) of the plate, 4 mL per well. See illustration for lysate layout in Orochem filters (FIG. 3).

Centrifuge lysates in filter plates, set on top of 24-well collection plates, at 980 g for 5 min. Discard filter plates. Lysate filtration ensures a smooth downstream affinity purification and helps avoid clogging IMCS tips.

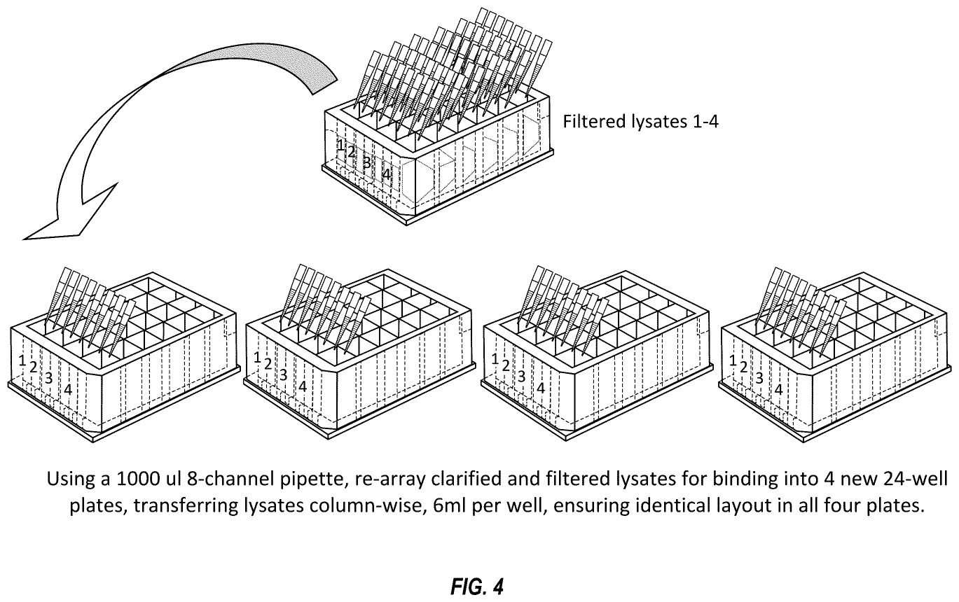

Using a 1000 μL 8-channel pipette, re-array clarified and filtered lysates for binding into four new 24-well plates, transferring lysates column-wise, 6 mL per well, ensuring identical layout in all four plates. See illustration for the layout of lysates for binding in 24-well plates (FIG. 4).

Prepare resin-filled IMCS tips by arraying them in a Hamilton 1 mL tip rack to match the layout of lysates in the 24-well plates. The amount of resin needed for an affinity purification can be estimated based on the resin binding capacity and the expected expression yield of the target protein. In this protocol, it is possible to use 50, 100, 150 or 200 μL resin per sample. To use 200 μL resin per purification, place 4 tips in rack positions, corresponding to the well for that sample in the 24-well plate. This protocol allows the use of different resins for different samples to match the resin with the purification tag of the target protein. For example, Ni-NTA resin, Sigma M2 anti-FLAG resin and Streptactin HC resin can be used for polyhistidine-, FLAG- and Strep-tagged proteins, respectively. We have evaluated Phynexus and IMCS tip columns and determined that both performed equally well in mid-scale protein affinity purification. It is important to note that the use of Phynexus tips requires different settings on Hamilton STAR from those for IMCS tips for proper method execution.

Prepare a 96-well plate with 300 μL Elution Buffer in wells corresponding to tip layout. The total elution volume for a purification performed with four IMCS tips will be equal to 1,200 μL.

Prepare 2 trays for blotting by assembling filter paper on top of the pad in an omnitray.

Turn on the 4° C. water cooler on Hamilton STAR to cool the deck that will hold samples during purification.

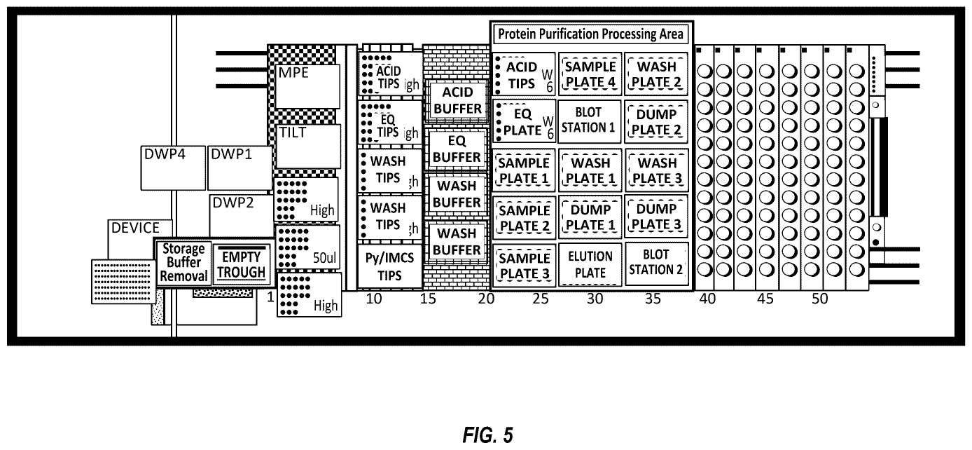

Prepare the Hamilton STAR™ deck by arranging the tips, buffers, plates and blotting stations as shown in FIG. 5.

### Materials:

Trough filled with water for dispensing storage buffer from IMCS tips
IMCS tips
Three racks of CO-RE tips for buffer transfer
Trough filled with Acid Buffer
Trough filled with Equilibration Buffer
Two troughs filled with Wash Buffer
96-well plate for Acid Buffer
96-well plate for Equilibration Buffer
Three 24-well plates for Wash Buffer
Three empty 24-well plates for dispensing used Wash Buffer
Two blotting stations (To prepare a blotting station, place a lint-free blotting media on top of a super absorbent polypropylene pad fitted into an omnitray)
Four 24-well plates containing filtered lysates, 6 mL per well
96-well plate with 300 μL Elution Buffer per well

Start IMCS mid-scale protein purification method on Hamilton STAR™ (see Support Protocol 2 for method details). After the Hamilton STAR™ method is complete, remove all material from the deck. Turn off the water cooler. Note: Daily and weekly maintenance is performed through the “Microlab STAR Maintenance & Verification” application provided by Hamilton Company. Additionally, the 96-probe Multi-Probe-Head (MPH) and 8-Channel pipetting modules are wiped down with water and clean, lint-free, dry cloth after each run.

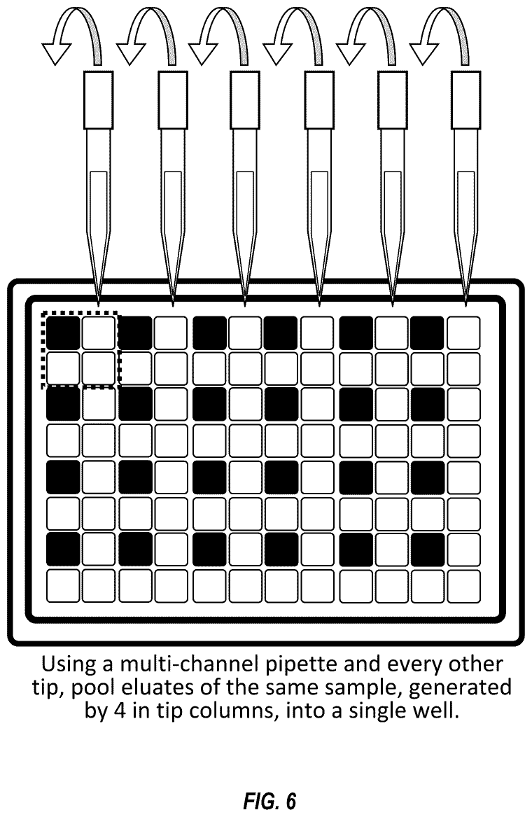

Pool eluates separately for each affinity purification sample. See illustration for eluate pooling (FIG. 6). Perform A280 measurements using a NanoDrop. For blank, use Elution Buffer without 3×FLAG peptide.

Prepare reduced samples for SDS-PAGE analysis by mixing 20 μL eluates with 3 μL of 10× reducing agent and 7 μL of 4×LDS running buffer in a PCR plate, while maintaining the same layout of samples as in the pooled elution plate. Heat samples at 95° C. for 5 minutes using a PCR thermocycler. Load 20 μL of sample and 7 μL of Prestained Protein standard on NuPAGE gel. Run in 1× NuPAGE™ MES SDS Running Buffer at 180V for 50 min. Running time can be adjusted based on the expected molecular weight of the proteins. For example, proteins smaller than 15 kDa should not be run for more than 47 minutes, while very large proteins that are greater than 200 kDa may require a longer running time for better resolution.

Using a gel knife, break open the gel cassettes and transfer the gels into trays filled with water. Rinse gels in water, stain in InstantBlue Coomassie stain for 1 hour while rocking gently on a rocking platform. De-stain in water for 2 hours while continuing to rock on a rocking platform.

Scan and label gels using Bio-RAD Image Lab™ software. Scanning is done on Bio-RAD Gel Doc™ EZ Imaging System using white tray by selecting Default protocol and Coomassie Blue as application. Image exposure is set for “Faint Bands” or manual exposure at 0.500 sec. Image analysis is done by selecting “Lane and Bands” tool as well as “Analyze Molecular Weight” features in Image Lab software Analysis tool box. Bands of interest are annotated using Annotation tools.

### Support Protocol 2

Automated method for affinity purification on Hamilton STAR™

Hamilton STAR™ IMCS mid-scale purification method as used in this

Example used the following steps:

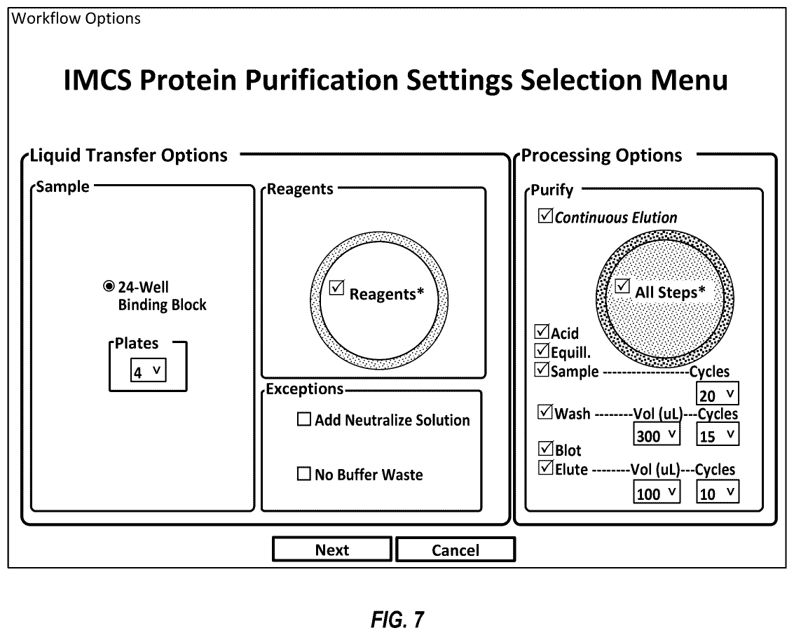

The method parameters, such as the number of sample binding cycles and the number of elution cycles, can be selected on the user interface (FIG. 7). The recommended method parameters are listed in Table 2. The acid addition step transfers 1 mL of Acid Buffer from the trough using Co-RE tips into the 96-well Acid Plate. The aspiration and dispense of buffer is performed at 250 and 400 [symbol] L per second, respectively. Note: 50 μL pre-aspiration of air is performed prior to each protein purification step. After all mixing cycles are performed and the final dispense with the 30 second wait time are completed, the 50 μL pre-aspiration is used as a blowout above the surface of the liquid.

The equilibration addition step transfers 1 mL of Equilibration Buffer from the trough using Co-RE tips into the 96-well Equilibration Plate. The aspiration and dispensing of buffer are done at 250 and 400 [symbol] L per second, respectively.

The wash buffer addition step transfers Wash Buffer, 1 mL per channel, twice into the 24-well Wash Plate using Co-RE tips. The transfer is repeated two more times for two additional wash plates. The aspiration of buffer is performed at 250 [symbol] L per second. The dispensing of the buffer is done at 400 [symbol] L per second.

Using the “MIStarIsCoreHeadSpecialTipPickup” tip property, storage buffer is dispelled into a trough, filled with water.

Acid wash step washes all resin tips with 800 μL of Acid Buffer by pipetting up and down once at a flow rate of 100 μL per second for aspiration and 30 μL per second for dispensing and includes a 30-second pause after the dispense. Note: The pipetting steps using resin-containing IMCS tips are performed at a significantly slower rate than the pipetting steps using Co-RE tips. This is done to reduce the pressure build-up in the IMCS tips. The change in aspiration and dispense speed for IMCS tips supports the dispersion and settling of resin during mixing. The faster aspiration provides more efficient resin distribution while the slower dispense speed provides proper resin settling. Additionally, the slower dispensing speed prevents resin from adhering to the wall of the tips.

Equilibration step washes all resin tips with 800 μL of Equilibration Buffer by pipetting up and down 3 times at a flow rate of 100 μL per second for aspiration and 30 μL per second for dispensing and includes a two-second pause between each aspirate and dispense cycle and a 30-second pause after the final dispense.

The sample binding step loads proteins onto tips by pipetting 700 μL up and down 20 times in each sample plate at a flow rate of 100 μL per second for aspiration and 15 μL per second for dispensing. This step includes a 2-second pause between each aspiration and dispense cycle and a 30-second pause after the final dispense. Note: The cell lysates are considerably more viscous than buffers used in this method, which requires an even further reduction in the rate of dispensing during the sample binding step.

Blotting step blots the tips onto absorbent paper after the final binding step.

The wash step washes all tips with Wash Buffer by transferring 300 μL of Wash Buffer 5 times from each of the three Wash plates, for a total of 15 transfers of 300 μL per tip. Flow rate of 100 μL per second is used for aspiration and 30 μL per second for dispense. This step utilizes a 2-second pause between eh aspirate and dispense cycle and a 30-second pause after the final dispense.

The elution step elutes proteins in Elution Buffer by aspirating and dispensing 250 μL 20 times at a flow rate of 100 μL per second for aspiration and 15 μL per second for dispensing and includes a 2-second pause between each aspirate and dispense cycle and a 30-second pause after the final dispense.

### Basic Protocol 4

### Size Exclusion Chromatography (SEC)

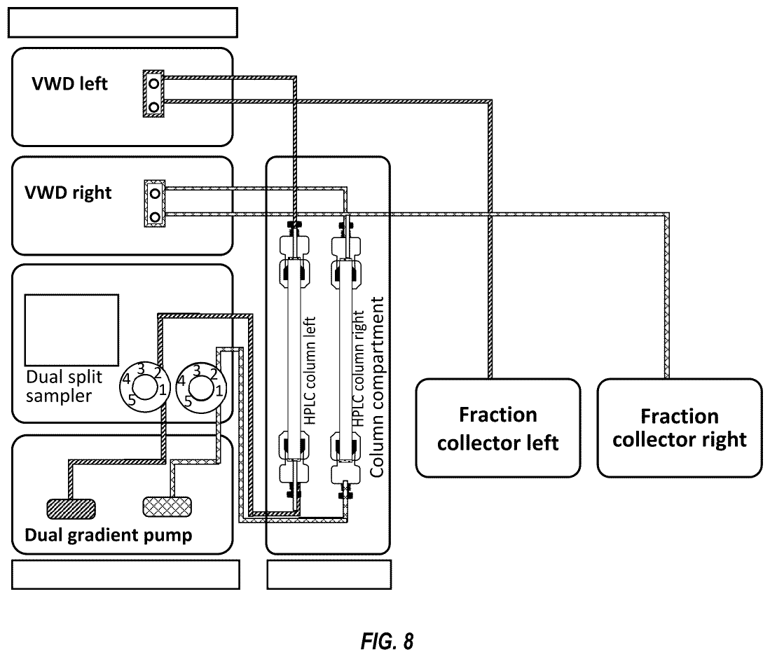

This protocol describes SEC, the second purification step that was used in the mid-scale workflow. The Thermo Fisher Vanquish™ Duo system (FIG. 8) and Phenomenex Yarra™ series column/TSK Super SW column are used for this protocol. The wavelength used was 280 nm for analyzing protein peaks.

### Materials

The following materials were used:
SEC Buffer (see recipe in Reagents and Solutions)
Millipore Amicon Ultra 0.5 centrifugal filters, Ultracel 3 kD (Cat. No. UFC500396)
Millipore Ultrafree centrifugal filters, Dura Free PVDF 0.22 μM (Cat. No. UFC306VOO)
Thermo Fisher Vanquish Duo HPLC system
Phenomenex column Yarra series 3 μM SEC-3000, LC column 300×4.6 mm (Cat. No. 00H-4513-EO)
TSKgel SuperSW3000 4.6 mm×30 cm (Cat. No. 0018675)
Waters BEH SEC Protein Standard mix (Cat. No. 186006518-1)
1× Phosphate Buffered Saline (PBS) (Invitrogen, 10×, Cat. No. AM9624)
Thermo Scientific™ WebSeal™ Well Plates, barcoded for Vanquish™ UHPLC Systems (Cat. No. 60180-P103B)
96 Well Microplate, Round Well, Barcoded
Thermo Scientific™ Plate seal (Cat. No. 60180-M146)
Thermo Scientific™ Abgene 96 well 2.2 mL Polypropylene Deep Well Storage Plate (Cat. No. AB0932)

### Protocol Steps for Size Exclusion Chromatography Using Thermo Fisher Vanquish Duo System

The following protocol steps were used:

Concentrate the eluates from affinity purification to ˜130 μL, using 3 kDa Amicon spin concentrators by centrifuging @13000 g for 20 minutes in a refrigerated centrifuge and filter using Amicon 0.2 μm filter tubes. Samples need to be concentrated as the Thermo Fisher Vanquish Flex model only provides configuration with sample loop size of 25 μL and 100 μL, thus the maximum amount of sample which can be loaded is 100 μL.

For higher MW proteins or complexes, a higher MW cut-off concentrator can be used. Some proteins tend to precipitate while concentrating and cannot be loaded on SEC. See Troubleshooting Table 1.

Prepare BEH Molecular Weight Standard mix by dissolving in 0.5 mL of PBS and filtering with an Amicon 0.2 μm filter. Load concentrated and filtered samples along with BEH Protein Standard mix in a 96 well ThermoFisher plate with barcode, seal the plate and place it on one of the racks in Autosampler (temperature set to 4° C.) on Vanquish Duo system (FIG. 8). Samples can be loaded on any other 96-well microplate, as long as it has round well bottoms. If plates from another vendor are used, please specify plate format in Autosampler on the Chromeleon console. Plate seals are optional and generally recommended to avoid evaporation of samples if kept in the Autosampler for more than 24 hours.

Follow steps provided in Support Protocol 3 for system operation and method set-up.

Equilibrate SEC column Phenomenex Yarra SEC 3000 or TSKgel Super SW3000 column (Column volume 5 mL) with SEC running buffer for 30 minutes at a flow rate of 0.3 mL/min. This column's recommended flow rate is 1 mL/min; however, to allow better resolution and avoid pressure build up, we recommend a flow rate of 0.3 mL/min. For purification of single proteins, the flow rate can be increased to 0.6 mL/min. TSKgel Super SW 3000 column can be used instead of Phenomenex Yarra SEC 3000 column.

Create a sequence of injections with the appropriate instrument method in Chromeleon console as described in Support Protocol 3. Stop monitoring baseline and start the sequence run. All 24 samples from affinity purifications can be loaded onto one plate for SEC purification. It takes about 33 minutes to complete one sample at a flow rate of 0.3 mL/min.

Collect fractions on the fraction collector in Thermo Scientific Abgene™ deep-well 96-well plates. Fraction collection mode can be chosen as “collection by peak” or “collection by time”. We have determined that “collection by peak” works better for us and collects fractions only when peak is identified (settings can be adjusted in the method). “Collection by time” collects fractions in between peaks and results in more than 100 fractions per sample. Alternatively, fraction collection can be turned off when only protein analytical characterization is needed. Fraction size can be adjusted by changing “Tube change duration” parameter on the Fraction Collector settings. We collect 40-50 μL fractions using a value of 4 sec for “Tube change duration” as shown in the Method script.

Analyze the chromatograms and identify peaks of interest to identify well numbers for fraction analysis by SDS-PAGE. Chromeleon data analysis provides an option of stacking or overlaying chromatograms to compare samples. We prefer to analyze chromatograms using the stack option.

Note down the fraction numbers and aliquot 20 μL of each fraction in Axygen 96 well plate for gel loading. Prepare reduced samples for SDS-PAGE analysis by mixing 20 μL of sample with 3 μL of 10× reducing agent and 7 μL of 4×LDS running buffer in a PCR plate. Seal the plate first with Aluminum foil seal and boil samples at 95° C. for 10 minutes using a PCR machine. Load 20 μl on Novex pre-made gel. Run at 180V for 50 min. Running time can be adjusted based on the expected molecular weight of the proteins. For example, proteins smaller than 15 kDa should not be run for more than 47 minutes, while very large proteins that are greater than 200 kDa may require a longer running time to achieve better resolution.

Rinse gels in water, stain in InstantBlue Coomassie stain for 1 hour by placing gel tray on rocker, and then de-stain in water for 2 hours while rocking on rocker.

Scan and label gels using Bio-RAD Image Lab™ software. Scanning is done on Bio-RAD Gel Doc™ EZ Imaging System using white tray by selecting Default protocol and Coomassie Blue as application. Image exposure is set for “Faint bands” or manual exposure at 0.500 sec. Image analysis is done by selecting “Lane and Bands” tool as well as “Analyze Molecular Weight” features in Image Lab software Analysis tool box. Bands of interest are annotated using Annotation tools.

Pool fractions in which the desired molecular weight proteins of interest are present.

Create data report. The proteins are ready for downstream characterization.

This purification method results in high quality purified proteins and multiprotein complexes. The yields vary depending on the expression level of target proteins. If no protein detected, please refer to Troubleshooting Table TI.

### Support Protocol 3

### Chromeleon 7 Operation on Vanquish Duo

Open Chromeleon 7 console on Windows™ computer interfaced with

Vanquish™ Duo system.

Open only Vanquish Left icon Vanq_Left if only the left system needs to be operated. Both Vanquish Left and Right systems can be operated at the same time by controlling from Vanq_Left or Vanq_Right. For simplicity, we will describe only the Vanq_Left system.

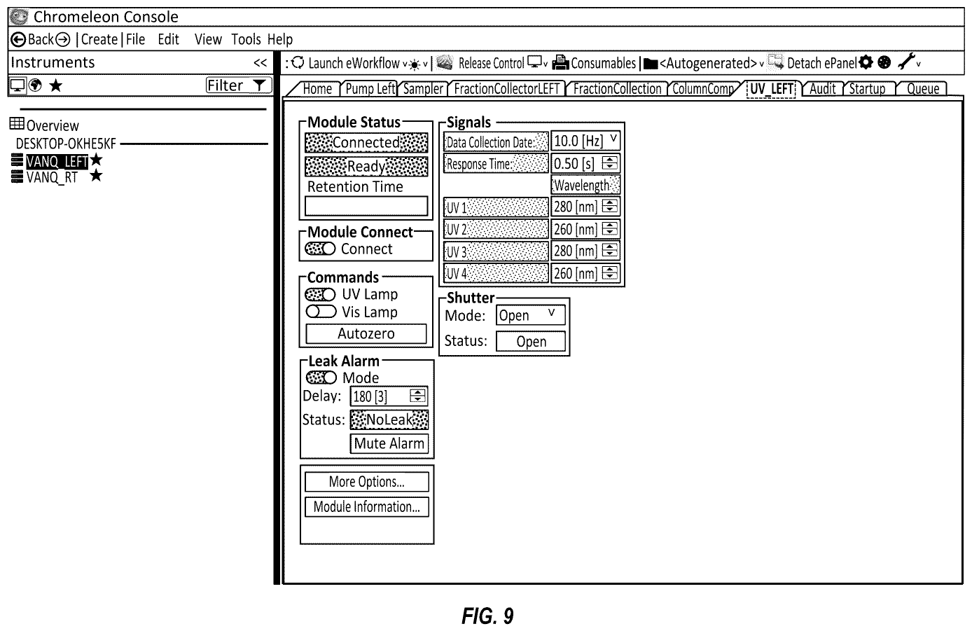

Open the “Instruments” tab on the left bottom corner as shown in the screenshot (FIG. 9). Turn on UV lamp and Vis lamp under UV Left tab by turning on toggle switch, which will turn green from gray in about 15 minutes.

Open shutter by selecting Open position from drop-down option under UV tab.

Connect the module and change the column compartment temperature to 10° C. under Column compartment tab.

Reset the Fraction Collector to fraction 1 under Fraction Collection tab.

Set the pump pressure to 350 psi in Pump_Left tab. This is calculated by adding maximum pressure allowed for the column (provided by manufacturer) and pressure of the HPLC system. If pump pressure showing higher than set value, please see Troubleshooting Table 1. If pump pressure is not increasing with increase of flow rate, there can be leaks in the system. Please refer to Troubleshooting Table 1.

Adjust the flow rate to 0.3 mL/min in Pump Left_tab. Alternatively, flow rate can be adjusted to 0.6 mL/min and maximum of 1 mL/min for Phenomenex Yarra series columns. We chose 0.3 mL/min for better resolution of our protein peaks.

Load SEC buffer onto the buffer station on the top stack, insert Left system A_L filter into the buffer, and close the cap. Open the Vanquish left pump valve by turning the knob 360°. This allows purging of the system at high flow rate without building up pressure. The default setting for purging is 5 mL/min with acceleration and deceleration of 0.1 mL/min. These settings may be adjusted under options tab. You should hear the sound of purging and rear seal wash piston moving.

After purging stops, close the left pump valve. If the valve is not closed, the motor will not turn on when you run the system.

Turn on the motor from the toggle switch and the system will start running at the set flow rate.

Connect the column to the valve and place it in column compartment. Column needs to be connected upside down as the direction of flow in this system is from bottom to top, i.e., pump is at the bottom and detectors are on the top of the stack.

Monitor baseline by selecting UV wavelength of 280 nm and pump pressure from the dialog box. A280 (Absorbance at 280 nm) will show under the fraction collection tab and pump pressure will show on the Pump Left tab. Absorbance at 260 nm (for nucleic acids detection) can also be monitored simultaneously but data collection slows down when two wavelengths are selected with this detector.

Equilibrate the Phenomenex Yarra SEC3000 column or TSK column (Column volume is 5 mL) with SEC running buffer for 30 minutes at flow rate of 0.3 mL/min.

Turn the baseline monitoring off after 30 minutes of equilibration.

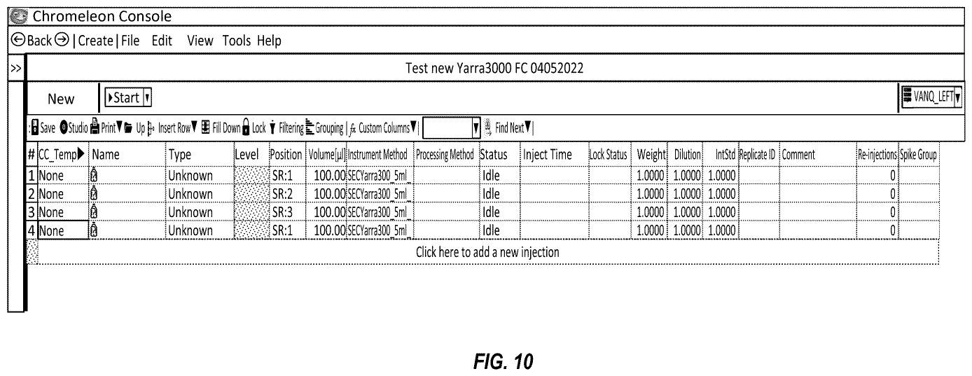

Open the “Data” Tab on the left bottom corner and start creating a method run. Open “Create New Sequence” from the Create icon on the top left corner (FIG. 10).

Add line items based on the number of samples.

Add name and position of sample in 96 well plate for each sample (e.g., for a sample in A12 well on a plate in green location, enter “G: A12”).

Select Instrument method for each sample. Instrument method can be set up once as shown below and used for all future purifications if the same column is used and no other parameters are changed.

Start the run and fraction collection.

Wash the column with buffer and deionized water after the run is completed for at least one column volume at a flow rate of 1 mL/min.

Disconnect the column and store at 4° C. HPLC lines are washed and maintained with 10% methanol wash every 2 months to avoid any contamination growth. Rear seal wash line is maintained active and operational with 10% methanol and re-filled every month to maintain high lifetime of pistons and pistons seals.

Reagents and solutions used in the method are as follows:

### Acid Wash Buffer (pH3.0)

100 mL of 1 M Glycine-HCl pH3.0 (100 mM final)
30 mL of 5 M NaCl (150 mM final)
870 mL water
Corrosive, must be stored with acids
Store at room temperature for up to 12 months

### Elution Buffer (pH7.5)

50 mL of 1 M Tris pH7.5 (50 mM final)
30 mL of 5 M NaCl (150 mM final)
100 mL of 50% glycerol (5% final)
125 mL of 2 M imidazole (250 mM final)
695 mL water
TCEP (1 mM final)
3×FLAG peptide (150 μg/mL final)
Roche EDTA-free Protein Inhibitor Cocktail (PIC) (1 tablet per 50 mL of buffer final)
Buffer made with Tris, NaCl, glycerol, and imidazole can be stored at 4° C. for up to 1 year. TCEP, 3×FLAG peptide and PIC must be added fresh prior to use.

### Equilibration and Lysis Buffer

50 mL of 1 M Tris pH7.5 (50 mM final)
30 mL of 5 M NaCl (150 mM final)
200 mL of 50% glycerol (10% final)
2 mL of 1M MgCl₂ (2 mM final)
5 mL of 2 M imidazole (10 mM final)
713 mL water
TCEP (1 mM final)
Benzonase (0.5 μL per 1 mL of buffer final)
Roche EDTA-free Protein Inhibitor Cocktail (PIC) (1 tablet per 50 mL of buffer final) Buffer made with Tris, NaCl, glycerol, imidazole and MgCl₂ can be stored at 4° C. for up to 1 year. TCEP, benzonase, and PIC must be added fresh prior to use.

### SEC Buffer

50 mL of 1 M Tris pH7.5 (50 mM final)
30 mL of 5 M NaCl (150 mM final)
920 mL water
TCEP (1 mM final)
Buffer made with Tris and NaCl can be stored at 4° C. for up to 1 year. TCEP must be added fresh prior to use.

### Wash Buffer

50 mL of 1 M Tris pH7.5 (50 mM final)
30 mL of 5 M NaCl (150 mM final)
100 mL of 50% glycerol (5% final)
10 mL of 2 M imidazole (20 mM final)
810 mL water
TCEP (1 mM final)
Roche EDTA-free Protein Inhibitor Cocktail (PIC) (1 tablet per 50 mL of buffer final)
Buffer made with Tris, NaCl, glycerol, and imidazole can be stored at 4° C. for up to 1 year. TCEP and PIC must be added fresh prior to use.

### Multi-Component Protein Expression

Many laboratories have built semi-automated high-throughput small scale expression analysis platforms to allow simultaneous analysis of hundreds of constructs with different tags, domains, and in multiple cell lines. This allows researchers to choose efficiently the best condition for expressing their proteins with good yields in most cases. However, this approach does not work very well for challenging poorly expressing proteins or multiprotein complexes. Hence, there was a need to build a medium scale protein expression and characterization platform that allows parallel and rapid triaging of challenging low-expressors and co-expressions in a high throughput manner. We built and developed this medium scale platform based on a small scale expression analysis platform (Kraft et al., 2019). It is an end-to-end semi-automated workflow from virus generation to protein production and enables delivery of 24 different proteins in a single purification run. It involves a two-step purification which includes affinity and size exclusion chromatography. This workflow, in addition to protein expression and characterization, has evolved to provide a few hundred micrograms of high quality purified proteins for further downstream applications. We have successfully explored many applications using proteins purified from this platform, including negative stain, High-Throughput Time Resolved Fluorescence (HTRF) assay, SPR, affinity pulldowns, enzymatic assays, and mass spectrometry analysis. Another potential application of this method is to screen for optimal buffer or co-expression ratios of different components to reconstitute soluble, stable protein complex.

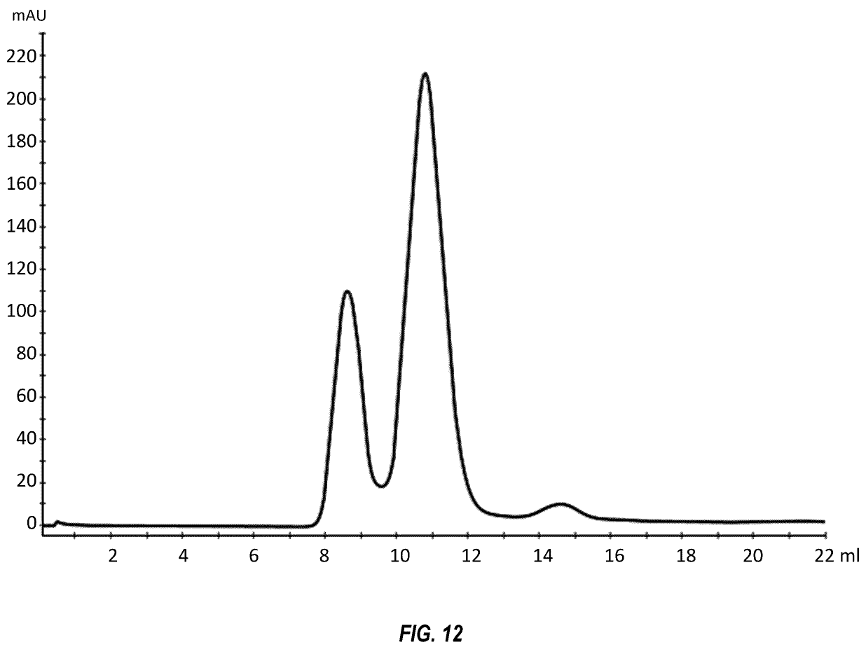

In this Example, we have focused on expression and purification of a multiprotein complex with 8 component subunits that was successfully used for negative stain application. The results described here using this protocol were followed up further for large scale expression and purification at 3 L scale. Large scale purification was performed using conventional batch mode of affinity purification with M2 anti-FLAG resin, ion exchange chromatography, and SEC. SEC analysis showed very similar mono-dispersive peak for the protein complex and resulted in about 0.3 mg of protein complex per liter of culture (FIG. 12). This purified protein was then used to solve a high resolution cryo-EM structure of the complex (not published). Overall, the end-to-end mid-scale protein screening platform enables parallel expression and purification of 24 samples or conditions and allows delivery of proteins in usable quantities for downstream applications.

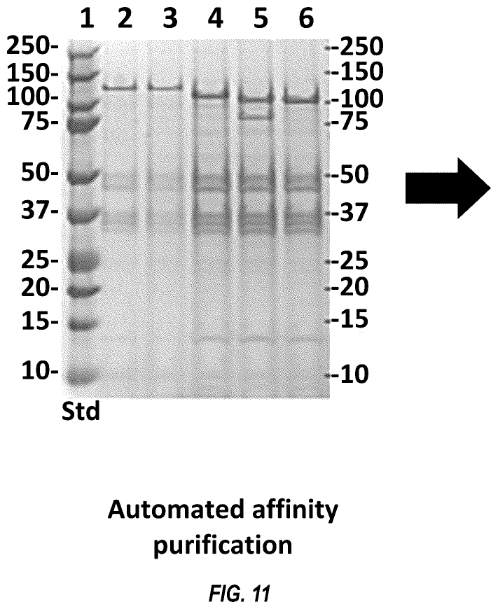

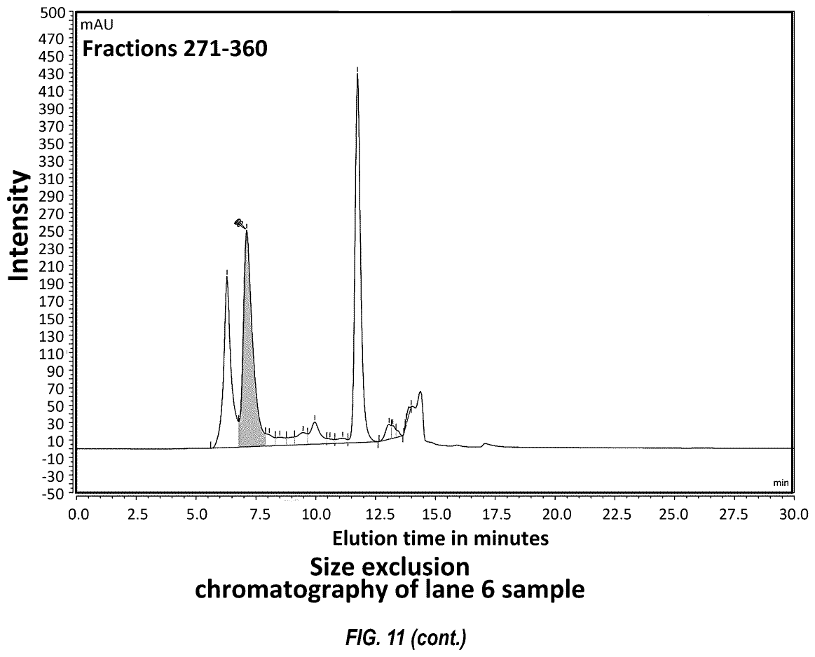

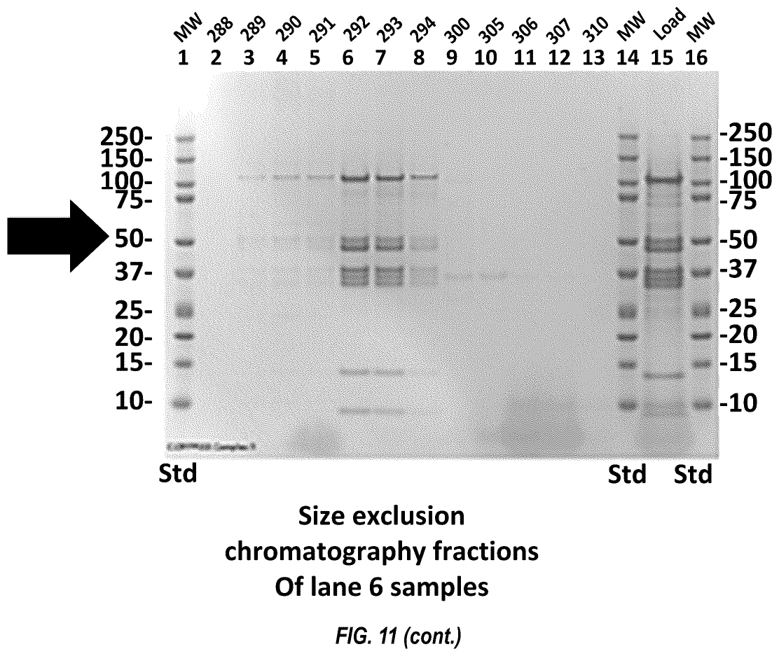

FIG. 11 shows one successful case study to express and purify a multiprotein complex from insect cells using the mid-scale platform. Protein complexes in insect cells were expressed using co-infection of multiple viruses each expressing one single protein subunit. Often, for large protein complexes that contain 4 or more subunits, co-infection of multiple viruses can lead to increased cell stress and uneven infection of cells, which can result in incomplete complex formation and/or low protein yields. To reduce the heterogeneity of viruses upon co-infection, multi-ORF constructs that express multiple proteins from a single virus can be used to improve complex stoichiometry and yields. In an effort to enable production of an 8-subunit protein complex (Proteins A-H), we designed 3 constructs with the following strategy:
1) The largest subunit (Protein A, 117.1 kDa) was N-terminally FLAG-tagged and expressed on its own vector under polyhedrin promoter. This was to enable screening numerous N-terminal truncations as part of a domain walking strategy to increase protein yields.
2) Proteins B-E (51.8, 6.1, 34.8, and 47.2 kDa) were generated as untagged proteins and placed on a separate vector backbone, with each ORF under the control of a polyhedrin promoter.
3) Proteins F-H (39.2, 36.4, 14.5) encoding three different ORFs were placed on a third vector with each ORF under individual polyhedrin promoter.

Viruses were generated from each of these three constructs with the protocol listed above and all three viruses expressing 8 different proteins were co-expressed with 1:1:1 ratio in Sf9 cells for generating biomass. Biomasses were harvested, cells were lysed and proteins were purified using affinity purification and size exclusion chromatography. The first SDS-PAGE gel of FIG. 11 shows results from affinity purification, where all 8 protein subunits of the complex were purified using M2 anti-FLAG resin in IMCS tips on Hamilton STAR™ workstation with automated INtip™ affinity purification method described above. All proteins of the complex were detected at the expected molecular weight, further purified, and analyzed for the oligomeric status/aggregation status of the complex on size exclusion chromatography using TSK column. The chromatogram showed a mono-disperse peak and the complex eluted at an elution time of 7.5 minutes that corresponds to elution volume of 2.25 mL. The second gel in FIG. 11 shows analysis of fractions collected from size exclusion chromatography and all 8 proteins of the complex were detected in the analyzed peak. Total yield of this complex was observed to be about 0.43 mg from 200 mL culture. Peak fractions were collected and analyzed by negative stain electron microscopy. Samples were evaluated qualitatively, looking at uniformity of particles in each micrograph. This helped us to identify the optimal domain boundary for protein A that resulted in a stable and well behaved protein complex, and that appeared amenable for structural studies.

TABLE 1
Troubleshooting guide for mid-scale expression and purification from insect cells

| Problem | Possible cause | Solution |
|---|---|---|
| GP64 staining less than 20% | Indicative of contamination | Clean up DNA with Zymo Clean |
| | | N′ Concentrate Kit and repeat |
| | | transfection |
| Cell viability is low after | Too much virus added | Add less virus |
| recommended incubation time | Expressed protein(s) is(are) | Consider Mammalian |
| | toxic to the cells | expression system |
| | The culture was incubated for | Harvest earlier |
| | too long | |
| Cell diameter remains small | No infection or insufficient | Regenerate virus or use more |
| after recommended | amount of virus added | if appropriate |
| incubation time | | |
| No bands on gel | No expression | if tip pipetting failure, repeat |
| | Tip pipetting failure | the purification with new tip |
| High background of impurities | Low expression level of target | Increase the number of wash |
| after affinity pull-down | protein(s) | steps or increase the |
| | Insufficient washing of the | concentration of imidazole in |
| | resin/insufficient | Wash Buffer for polyhistidine- |
| | concentration of imidazole in | tagged protein purification |
| | Wash Buffer for polyhistidine- | |
| | tagged protein purification | |
| Protein shows precipitation | Biophysical property of the | Centrifuge and analyze the SEC |
| after concentration | protein | input by SDS-PAGE to see if |
| | | enough protein is left in the |
| | | supernatant |
| High pressure on HPLC system | Flow rate is too high under | Reduce flow rate |
| | given column settings | |
| Leak in HPLC system | Loose screws | Check ingoing and outgoing |
| | | valves to the column and if |
| | | leak is from there, tighten the |
| | | screws |
| No peaks on chromatogram or | Protein aggregated or stuck on | Analyze void peak fractions |
| void peak only | column | Continue running buffer |
| | | through the column while |
| | | monitoring UV280 |

TABLE 2
Automated affinity purification parameters
Method Parameters for Affinity Purification with 1 mL IMCS tips

| | | Reagent | | | Mixing | | |
|---|---|---|---|---|---|---|---|
| | | Transfer | | Mixing | Asp/Dis | Preaspiration/ | Preaspiration/ |
| | | Volume | Mixing | Volume, | Speed, | Blowout | Blowout Asp/Dis |
| Step | Buffer | (μL) | Cycles* | μL | μL/s | Volume, μL | Speed, μL/s |
| 1 | Acid | 1000 | 1 | 800 | 100/30 | 50 | 20/20 |
| 2 | Equilibration | 1000 | 3 | 800 | 100/30 | 50 | 20/20 |
| 3 | Sample | NA | 20 | 700 | 100/15 | 50 | 20/20 |
| 4 | Wash 1 | 2000 | 5 | 300 | 100/30 | 50 | 20/20 |
| 5 | Wash 2 | 2000 | 5 | 300 | 100/30 | 50 | 20/20 |
| 6 | Wash 3 | 2000 | 5 | 300 | 100/30 | 50 | 20/20 |
| 7 | Elution 1 | NA | 20 | 250 | 100/15 | 50 | 20/20 |

*On the last mixing cycle, a 30 second hold time is added after the aspiration step

### Further Comments

### High-Titer Virus Generation

Baculovirus generation in this protocol relied on successful in vitro recombination of transfer plasmid with linearized baculovirus DNA BestBac™ (Expression systems). The efficiency of homologous recombination should be between 90-100%, but can vary depending on the linearized vector used (Kitts et al., 1990). There are many commercially available linearized bacmids (BaculoGold™, BD Biosciences; FlashBAC, Oxford Expression Technologies; BacPAK™, Clontech) that use the method of homologous recombination in vitro. However, the final virus stock unavoidably contains a mixture of parental and recombinant viruses. Virus should be of high quality and high titer to proceed with expression at mid-scale. To avoid generating a high percentage of non-recombinant virus, the virus stock should not be amplified past P3 stage. Although we did not quantify the viral titer, we used the gp64 assay to determine the percentage of cells infected with virus particles (Kitts & Green, 1999). Virus stock with titer less than 70% gp64 staining will result in non-productive infection and lower yields and therefore, we recommend repeating cotransfection to regenerate the virus.

### Insect Cell Infection

Insect cells Sf9 and T.ni should be healthy to have productive infections with viruses and should have a diameter of 15.5 and 19 μm, for Sf9 and T.ni cells, respectively, as measured by Vi-CELL BLU. Culture viability should be >=95% to proceed. If cells are of low viability or of larger diameter, fresh cells should be thawed and passaged for infections. Productive infection of cells is also measured using Vi-CELL counters. Cells are harvested for purification only if cell diameter is greater than 17 or 21 μm, for Sf9 and T.ni, respectively, and viability is between 50 and 85% respectively. Otherwise, the infection process is repeated.

### Efficient Cell Lysis and Filtration

Cells need to be completely lysed using appropriate lysis methods as described above and lysate should be filtered to remove debris completely. There are numerous physical and chemical methods to lyse cells, including osmotic shock, freeze-thawing, Dounce homogenization, sonication, and detergent-based lysis. Eukaryotic cells have a higher amount of nucleic acids than bacteria, and as a result, a nuclease (benzonase) should be included to reduce viscosity of the cell lysate. We found that bead-based lysis provides complete and efficient lysis of insect cells in a higher throughput manner. Cellular debris has to be clarified either through high-speed centrifugation or depth microfiltration system. Orochem's filtration plate is a depth microfiltration system that consists of a filter with an open-pore structure to remove cells and cell debris; and a filter-aid material with a tighter pore structure to remove colloidal matter simultaneously to deliver particulate-free feed for down-stream processes. If depth filtration is not available, then high speed centrifugation (18,000 g for 30 minutes) should be employed to clarify the lysates before purification.

### Buffer for Purification

Buffer for lysis, wash and elution for affinity chromatography should be chosen carefully depending on protein isoelectric point and aggregation properties. Most commonly used buffers for purification are Tris- or HEPES-based. Salt concentration should be kept between 100-150 mM NaCl for purifying multiprotein complexes as higher salt concentration can interfere with ionic interactions between different components of protein complexes. Salt concentration can be adjusted to 300 mM for purifying single proteins, as higher salt concentration results in better purity. A reducing agent such as DTT or TCEP should be added for purifying intracellular proteins. We include 5-10% glycerol in our buffers, as glycerol offers stability to the proteins.

### Affinity Purification

In addition to choosing the right buffer, it is important to choose the right resin type for purification and optimize the automation method for a given resin. Resins can have very different binding capacities. For example, the M2 anti-FLAG and Ni-NTA Superflow™ resins that we use have a binding capacity of 0.6 mg/mL and 20 mg/mL, respectively, and thus the number of IMCS tips should be adjusted accordingly to the expected expression level of each sample. Some resins have higher or lower rate of binding target protein, and thus the automated purification method should be adjusted accordingly for the number of sample binding and elution cycles.

### Size Exclusion Chromatography

We tested different chemistry columns with different sizes and found that the 5 mL columns from Phenomenex Yarra™ series and TSK column with dimensions of 300×4.6 mm worked well for separation of protein complexes purified from 200 mL scale. The column has tightly packed inert silica-based material with 290 Å pore size and 3 μm particle size with separation range of 5-700 kDa. Another advantage of these columns is that they are compatible with HPLC and UHPLC systems and allow a fast flow rate of 1 mL/min and can withstand 300 bar pressure.

### Example 2: Mid-Scale Production of Proteins from Mammalian Cell Culture

A Histidine-tagged membrane protein was co-expressed with and without one or both of two cytoplasmic protein chaperones in several different flasks, each with a volume of 30 mL each in EXPi293 cells transfected with appropriate plasmids for expression on day zero at 37° C. On the following day 1, the temperature was maintained at 37° C. in some of the flasks, while being dropped to 30° C. in others. On day 4, parallel affinity purification of the protein was performed with a Ni-IMAC nickel column and the yield of the protein and chaperone proteins was assessed for each individual sample, allowing for rapid comparison of different purification conditions (temperature and presence of one or both chaperone proteins).

### Example 3: Mid-Scale Purification of a Protein Complex

Several different protein purifications were run in parallel at mid-scale for purification of an E3 ubiquitin ligase protein complex from Sf9 insect cells in order to determine optimal conditions for protein expression and complex formation. A FLAG tag was utilized to pull down the protein in parallel, followed by size exclusion chromatography and electrophoresis analysis. The substrate recognition protein of the complex showed no expression by itself, while co-expression with other members of the complex and a chaperone protein enabled reconstitution of a stable E3 ligase complex. This example illustrated that parallel mid-scale protein purifications can assist in rapidly determining optimal conditions for purifying a protein complex from cell culture.

### Literature Cited

Bahia, D., Cheung, R., Buchs, M., Geisse, S., & Hunt, I. (2005). Optimisation of insect cell growth in deep-well blocks: development of a high-throughput insect cell expression screen. Protein Expr Purif, 39 (1), 61-70. doi:10.1016/j.pep.2004.08.020
Chambers, S. P., Austen, D. A., Fulghum, J. R., & Kim, W. M. (2004). High-throughput screening for soluble recombinant expressed kinases in Escherichia coli and insect cells. Protein Expr Purif, 36 (1), 40-47. doi:10.1016/j.pep.2004.03.003
Esposito, D., Garvey, L. A., & Chakiath, C. S. (2009). Gateway cloning for protein expression. Methods Mol Biol, 498, 31-54. doi:10.1007/978-1-59745-196-3 3
Fernandez, F. J., & Vega, M. C. (2016). Choose a Suitable Expression Host: A Survey of Available Protein Production Platforms. Adv Exp Med Biol, 896, 15-24. doi:10.1007/978-3-319-27216-0 2
Festa, F., Steel, J., Bian, X., & Labaer, J. (2013). High-throughput cloning and expression library creation for functional proteomics. Proteomics, 13 (9), 1381-1399. doi:10.1002/pmic.201200456
Gecchele, E., Merlin, M., Brozzetti, A., Falorni, A., Pezzotti, M., & Avesani, L. (2015). A comparative analysis of recombinant protein expression in different biofactories: bacteria, insect cells and plant systems. J Vis Exp (97). doi:10.3791/52459
Gileadi, O., Burgess-Brown, N. A., Colebrook, S. M., Berridge, G., Savitsky, P., Smee, C. E., . . . Pantic, N. H. (2008). High throughput production of recombinant human proteins for crystallography. Methods Mol Biol, 426, 221-246. doi:10.1007/978-1-60327-058-8_14
Hunt, I. (2005). From gene to protein: a review of new and enabling technologies for multi-parallel protein expression. Protein Expr Purif, 40 (1), 1-22. doi:10.1016/j.pep.2004.10.018
Kates, P. A., Tomashek, J. J., Miles, D. A., & Lee, L. A. (2020). Parallel sample processing using dispersive INtip micro-purification on programmable multichannel pipettes. Biotechniques, 68 (3), 148-154. doi:10.2144/btn-2019-0140
Abdulrahman, W., Radu, L., Garzoni, F., Kolesnikova, O., Gupta, K., Osz-Papai, J., . . . Poterszman, A. (2015). The production of multiprotein complexes in insect cells using the baculovirus expression system. Methods Mol Biol, 1261, 91-114. doi:10.1007/978-1-4939-2230-7_5
Jarvis, D. L. (2009). Baculovirus-insect cell expression systems. Methods Enzymol, 463, 191-222. doi:10.1016/S0076-6879 (09) 63014-7
Kates, P. A., Cook, J. N., Ghan, R., Nguyen, H. J., Sitasuwan, P., & Lee, L. A. (2023). Incorporation of automated buffer exchange empowers high-throughput protein and plasmid purification for downstream uses. SLAS Technol. doi:10.1016/j.slast.2023.01.005
Kates, P. A., Tomashek, J. J., Miles, D. A., & Lee, L. A. (2020). Parallel sample processing using dispersive INtip micro-purification on programmable multichannel pipettes. Biotechniques, 68 (3), 148-154. doi:10.2144/btn-2019-0140
Osz-Papai, J., Radu, L., Abdulrahman, W., Kolb-Cheynel, I., Troffer-Charlier, N., Birck, C., & Poterszman, A. (2015). Insect cells-baculovirus system for the production of difficult to express proteins. Methods Mol Biol, 1258, 181-205. doi:10.1007/978-1-4939-2205-5_10
Shi, X., & Jarvis, D. L. (2007). Protein N-glycosylation in the baculovirus-insect cell system. Curr Drug Targets, 8 (10), 1116-1125. doi:10.2174/138945007782151360
Kimple, M. E., Brill, A. L., & Pasker, R. L. (2013). Overview of affinity tags for protein purification. Curr Protoc Protein Sci, 73, 9 9 1-9 9 23. doi:10.1002/0471140864.ps0909s73
Kitts, P. A., Ayres, M. D., & Possee, R. D. (1990). Linearization of baculovirus DNA enhances the recovery of recombinant virus expression vectors. Nucleic Acids Res, 18 (19), 5667-5672. doi:10.1093/nar/18.19.5667
Kitts, P. A., & Green, G. (1999). An immunological assay for determination of baculovirus titers in 48 hours. Anal Biochem, 268 (2), 173-178. doi:10.1006/abio. 1998.3042
Kitts, P. A., & Possee, R. D. (1993). A method for producing recombinant baculovirus expression vectors at high frequency. Biotechniques, 14 (5), 810-817. Retrieved from https://www.ncbi.nlm.nih.gov/pubmed/8512707
Kost, T. A., Condreay, J. P., & Jarvis, D. L. (2005). Baculovirus as versatile vectors for protein expression in insect and mammalian cells. Nat Biotechnol, 23 (5), 567-575. doi:10.1038/nbt1095
Kraft, E., Franke, Y., Heeringa, K., Shriver, S., Zilberleyb, I., Kugel, C., . . . Bowman, K. (2019). Semiautomated Small-Scale Purification Method for High-Throughput Expression Analysis of Recombinant Proteins. Methods Mol Biol, 2025, 51-68. doi:10.1007/978-1-4939-9624-7 3
Lesley, S. A. (2009). Parallel methods for expression and purification. Methods Enzymol, 463, 767-785. doi:10.1016/S0076-6879 (09) 63041-X
Marsischky, G., & LaBaer, J. (2004). Many paths to many clones: a comparative look at high-throughput cloning methods. Genome Res, 14 (10B), 2020-2028. doi:10.1101/gr.2528804
Murphy, C. I., & Piwnica-Worms, H. (2001). Overview of the baculovirus expression system. Curr Protoc Protein Sci, Chapter 5, Unit5 4. doi:10.1002/0471140864.ps0504s00
Nettleship, J. E., Assenberg, R., Diprose, J. M., Rahman-Huq, N., & Owens, R. J. (2010). Recent advances in the production of proteins in insect and mammalian cells for structural biology. J Struct Biol, 172 (1), 55-65. doi:10.1016/j.jsb.2010.02.006
Snead, K., Wall, V., Ambrose, H., Esposito, D., & Drew, M. (2022). Polycistronic baculovirus expression of SUGT1 enables high-yield production of recombinant leucine-rich repeat proteins and protein complexes. Protein Expr Purif, 193, 106061. doi:10.1016/j.pep.2022.106061
Volkman, L. E., & Goldsmith, P. A. (1988). Resistance of the 64K protein of budded Autographa californica nuclear polyhedrosis virus to functional inactivation by proteolysis. Virology, 166 (1), 285-289. doi:10.1016/0042-6822 (88) 90176-6

What is claimed is:

1. A method for purifying one or more polypeptides from a plurality of mammalian cell culture samples, insect cell culture samples, or bacterial cell culture samples, comprising:
(a) growing a plurality of mammalian, insect, or bacterial cell culture samples expressing one or more polypeptides for purification at a scale of 20-500 mL;
(b) lysing the cells of the cell culture samples, wherein parts (a) and (b) for each of the plurality of cell culture samples are conducted in the same container;
(c) centrifuging the lysed cell culture samples and collecting a plurality of supernatant samples from the cell culture samples;
(d) clarifying the plurality of supernatant samples by depth filtration;
(e) placing the plurality of clarified supernatant samples into wells of a multi-well plate, wherein the supernatant samples have a volume of 2-30 mL per well; and
(f) subjecting the plurality of supernatant samples in the wells of the multi-well plate to affinity chromatography conducted with an affinity matrix in a pipette tip and placing the eluate from the chromatography into wells of a multi-well plate; wherein parts (e) and (f) are conducted on the plurality of samples in parallel.

2. The method of claim 1, wherein the following parts the method are automated: part (f), or parts (e) and (f).

3. The method of claim 1, wherein all of parts (a)-(f) are performed in parallel.

4. The method of any one of claims 1-3, wherein the plurality of clarified supernatant samples are placed into wells of the multi-well plate at a volume of 2-30 mL per well.

5. A method for purifying one or more polypeptides starting from a plurality of clarified cell lysis supernatant samples in a multi-well plate, which were obtained from a plurality of cell culture samples by a process comprising:
(a) growing a plurality of mammalian, insect, or bacterial cell culture samples expressing one or more polypeptides for purification at a scale of 20-500 mL,
(b) lysing the cells of the cell culture samples, wherein parts (a) and (b) for each of the plurality of cell culture samples are conducted in the same container;
(c) centrifuging the lysed cell culture samples and collecting a plurality of supernatant samples from the cell culture samples;
(d) clarifying the plurality of supernatant samples by depth filtration;
(e) placing the plurality of clarified supernatant samples into wells of the multi-well plate, wherein the supernatant samples have a volume of 2-30 mL per well;
the method comprising: (f) subjecting the supernatant samples obtained from the process of (a)-(e) to affinity chromatography conducted with an affinity matrix in a pipette tip and placing the eluate from the chromatography into wells of a multi-well plate; wherein, the method is conducted on the plurality of clarified supernatant samples in parallel, and optionally wherein the method is automated.

6. A method for affinity purification of polypeptides from a plurality of cell culture supernatant samples expressing one or more polypeptides for purification, comprising:
obtaining a multi-well plate comprising a plurality of clarified cell culture supernatant samples in wells of the multi-well plate at a volume of 2-30 mL per well, and subjecting the plurality of clarified cell culture supernatant samples to affinity chromatography conducted with an affinity matrix in a pipette tip and placing the eluate from the chromatography into wells of a second multi-well plate; wherein, the method is conducted on the plurality of supernatant samples in parallel, optionally wherein the method is automated,
wherein the plurality of cell culture supernatant samples were obtained from mammalian, insect, or bacterial cell culture samples expressing one or more polypeptides, which were grown at a scale of 20-500 mL and subjected to lysis, centrifugation, and clarification, wherein growth and lysis of the cells were conducted in the same container.

7. The method of any one of claims 1-6, wherein the method further comprises:
(g) conducting size exclusion chromatography (SEC) on the eluate of the affinity chromatography; and
(h) fractionating polypeptides from the SEC into wells of a multi-well plate, optionally wherein one or both of parts (g) and (h) is automated.

8. The method of claim 7, wherein parts (g) and (h) are automated.

9. The method of any one of claims 1-8, wherein clarified supernatant sample corresponding to a single cell culture sample is placed into more than one well of the multi-well plate.

10. The method of any one of claims 1-9, wherein clarified supernatant samples from different cell culture samples are placed into different wells of the multi-well plate.

11. The method of any one of claims 1-10, wherein from 8 to 96 clarified supernatant samples are processed in parallel, such as from 8 to 48, from 8 to 24, from 12 to 48, or from 12 to 24.

12. The method of any one of claims 1-11, wherein cells are lysed by addition of glass beads with shaking and/or wherein the cells are not lysed by sonication.

13. The method of any one of claims 1-12, wherein the plurality of cell culture samples are grown at a scale of 30-250 mL, 50-250 mL, 30-200 mL, 50-200 mL, or 100-200 mL.

14. The method of any one of claims 1-13, wherein the pipette tip comprising the affinity matrix has a volume of 0.5 to 2 mL, such as from 1 to 2 mL, or 0.5 to 1.5 mL, or 0.5 mL, or 1 mL, or 1.5 mL, or 2 mL.

15. The method of any one of claims 1-14, wherein the affinity matrix in the pipette tip has a bed volume of from 30 to 100 μL, such as from 30 to 70 μL, or from 40 to 50 μL, or 30 μL, 40 μL, 50 μL, 60 μL, 70 μL, or 100 μL.

16. The method of any one of claims 1-15, wherein the polypeptides are tagged with a polyhistidine, FLAG, streptavidin, glutathione-S-transferase (GST), or maltose binding protein (MBP) tag, and wherein the affinity matrix recognizes the tag.

17. The method of any one of claims 7-16, wherein the method comprises parts (g) and (h), and wherein the SEC chromatography is performed on an SEC matrix comprising particles with pore size between 140 and 500 Angstroms, and/or with a particle size of 3 to 5 microns, and/or with a molecular weight range of 5 to 700 kDa.

18. The method of any one of claims 1-17, wherein the method further comprises performing structural or functional analysis on the purified polypeptides, such as cryoelectron microscopy, mass spectrometry, protein-protein interaction assays such as surface plasmon resonance, or homogeneous time resolved fluorescence assays.

19. The method of any one of claims 1-18, wherein the one or more polypeptides comprise recombinant protein complexes.

20. The method of any one of claims 1-19, wherein the one or more polypeptides do not comprise antibodies or antibody subunits.

21. The method of any one of claims 1-20, wherein the cell culture samples are insect cell culture samples, such as Sf9 or T.ni cell culture samples.

22. The method of any one of claims 1-21, wherein the cell culture samples are mammalian cell culture samples.
